(19) United States
(12) Patent Application Publication
(10) Pub. No.: US 2012/0190656 A1
(43) Pub. Date: Jul. 26, 2012

(54) METHODS FOR SELECTING THERAPIES TO IMPROVE HDL CHOLESTEROL AND TRIGLYCERIDE LEVELS

(76) Inventors: Brian H. Shirts, Salt Lake City, UT (US); Steven C. Hunt, Salt Lake City, UT (US); Paul N. Hopkins, Salt Lake City, UT (US)

(21) Appl. No.: 13/331,927

(22) Filed: Dec. 20, 2011

Related U.S. Application Data
(60) Provisional application No. 61/425,541, filed on Dec. 21, 2010.

(51) Int. Cl. A61K 31/59; A61P 3/06; C12Q 1/68

(52) U.S. Cl. 514/167; 435/6.11

(57) ABSTRACT

Disclosed herein are diagnostic methods for determining whether subjects will be responsive to vitamin D therapy. The methods involve the detection of one or more single nucleotide polymorphisms in a test sample, in combination with other clinical factors. The tests are suitable for diagnosing and determining a treatment regime for patients having or suspected of having a cardiovascular disease, including hyperlipidemia or hypertriglyceridemia.

### CROSS-REFERENCE TO RELATED APPLICATIONS

The present application claims the benefit of U.S. Provisional Application No. 61/425,541, filed on Dec. 21, 2010, the entire contents of which are incorporated herein by reference in its entirety.

### TECHNICAL FIELD

The present disclosure relates generally to identifying patient populations for the treatment and management of cardiovascular disease, including hyperlipidemia and hypertriglyceridemia. In particular, the present disclosure relates to methods for analyzing genetic profiles of individuals harboring single nucleotide polymorphisms at specific loci, and administering suitable treatments therefor.

### BACKGROUND

The following discussion of the background is merely provided to aid the reader in understanding the present disclosure and is not admitted to describe or constitute prior art.

High density lipoprotein cholesterol (HDL) is a major independent predictor of cardiovascular disease. Genome-wide studies indicate that more than 40 genes are associated with HDL. See, e.g., Teslovich et al., Biological, clinical and population relevance of 95 loci for blood lipids. Nature. Vol. 466: 707-713 (2010). Genetic variants of these genes, however, account for less than 30% of HDL-level associated heritability. Id. In particular, the APOA1 and cholesteryl ester transfer protein (CETP) loci have been linked to HDL, total cholesterol, and triglyceride levels in patients. The APOA1 locus consists of 4 genes: APOA1; APOC3; APOA4; and APOA5, which are typically referred to as APOA1-APOA5, and encode for various proteins including Apolipoprotein A1 (Apo-A1), i.e., the major component of HDL particles. Despite knowledge of the basic biology of apolipoproteins, the identity and functional mechanisms of APOA1-APOA5 polymorphisms are not clearly defined.

Individuals harboring various genetic aberrations may be more or less responsive to hypolipidemic or hypotriglyceridemic drugs, including various nutrients such as vitamin D. Thus, genetic factors present at diagnosis are potential biomarkers which can implicate treatment protocols. Consequently, identification of individuals that may be responsive or resistant to particular therapies would improve their medical assessment at the time of a diagnosis.

### SUMMARY

In one aspect, the present disclosure generally describes methods for predicting or identifying whether a subject will respond to vitamin D therapy by determining an allelic genotype at one or more loci selected from rs10466589, rs10488699, rs10750097, rs11216103, rs11823543, rs11825181, rs12272004, rs12279373, rs12280724, rs12285095, rs12286037, rs12287066, rs12292921, rs12294259, rs28927680, rs3135506, rs35120633, and rs964184, in a sample from the subject, wherein a polymorphism at the one or more loci is an indication of the subject's responsiveness to the vitamin D therapy. In one embodiment, the polymorphism is in linkage disequilibrium with one or more additional polymorphisms at the one or more loci.

In one embodiment, the allelic genotype is an APOA1-APOA5 allelic genotype. In one embodiment, the allelic genotype is an APOA1-APOA5 allelic genotype at the rs3135506 loci in the sample from the subject, and the polymorphism is a G/C or C/C polymorphism. In one embodiment, the G/C or C/C polymorphism indicates that the vitamin D can increase the subject's HDL level compared to a reference level. In one embodiment, the reference level is the level of HDL in a sample from an individual without the G/C or C/C genotype.

In one embodiment, the allelic genotype is an APOA1-APOA5 allelic genotype at the rs10750097 loci in the sample from the subject, and the polymorphism is an A/G or G/G polymorphism. In one embodiment, the A/G or G/G polymorphism indicates that the vitamin D can increase the subject's HDL level compared to a reference level. In one embodiment, the reference level is the level of HDL in a sample from an individual without the A/G or G/G genotype.

In one embodiment, the increase is at least a 10% increase. In one embodiment, the G/C or C/C polymorphism indicates that the vitamin D can decrease the subject's triglyceride level compared to a reference level. In one embodiment, the reference level is the triglyceride level in a sample from an individual without the G/C or C/C polymorphism. In one embodiment, the decrease is at least a 10% decrease. In one embodiment, individuals without the G/C or C/C polymorphism have a G/G genotype at the rs3135506 loci.

In one embodiment, the A/G or G/G polymorphism indicates that the vitamin D can decrease the subject's triglyceride level compared to a reference level. In one embodiment, the reference level is the triglyceride level in a sample from an individual without the G A/G or G/G polymorphism. In one embodiment, the decrease is at least a 5% decrease. In one embodiment, individuals without the A/G or G/G polymorphism have aa A/A genotype at the rs10750097 loci.

In one embodiment, the methods further include administering the vitamin D to the subjects with the polymorphic allele at the one or more loci. In one embodiment, the vitamin D is administered from 10-10,000 IU. In one embodiment, the vitamin D is vitamin D2 or vitamin D3. In one embodiment, the subject is a human patient having or suspected of having hyperlipidemia or hypertriglyceridemia. In one embodiment, the determining comprises genotyping the sample using PCR.

In one aspect, the present disclosure describes method for increasing HDL in a subject by analyzing a sample from the subject to determine whether the subject will be responsive to vitamin D therapy based on results obtained from the sample, wherein a polymorphism at one or more loci selected from the group consisting of rs10466589, rs10488699, rs10750097, rs11216103, rs11823543, rs11825181, rs12272004, rs12279373, rs12280724, rs12285095, rs12286037, rs12287066, rs12292921, rs12294259, rs28927680, rs3135506, rs35120633, and rs964184, is indicative of the subject's responsiveness to the vitamin D therapy, and wherein the vitamin D can increase the HDL level in the subjects having the polymorphism compared to a reference level; and administering the vitamin D to the subjects having the polymorphism.

In one embodiment, the polymorphism is an APOA1-APOA5 polymorphism. In one embodiment, the polymorphism is G/C or C/C polymorphism at the rs3135506 locus. In one embodiment, the polymorphism is in linkage disequilibrium with one or more additional polymorphisms at the one or more loci. In one embodiment, the vitamin D is administrated from 10-10,000 IU. In one embodiment, the reference level is the level of HDL in individuals without the polymorphism at the one or more loci. In one embodiment, the increase is at least a 10% increase. In one embodiment, the sample is a body fluid sample. In one embodiment, the subject is a human patient having or suspected of having hyperlipidemia. In one embodiment, the analyzing comprises genotyping the sample using PCR.

In one embodiment, the polymorphism is an APOA1-APOA5 polymorphism. In one embodiment, the polymorphism is A/G or G/G polymorphism at the rs10750097 locus. In one embodiment, the polymorphism is in linkage disequilibrium with one or more additional polymorphisms at the one or more loci. In one embodiment, the vitamin D is administrated from 10-10,000 IU. In one embodiment, the reference level is the level of HDL in individuals without the polymorphism at the one or more loci. In one embodiment, the increase is at least a 10% increase. In one embodiment, the sample is a body fluid sample. In one embodiment, the subject is a human patient having or suspected of having hyperlipidemia. In one embodiment, the analyzing comprises genotyping the sample using PCR.

In one aspect, the present disclosure describes methods for decreasing triglyceride levels in a subject by analyzing a sample from the subject to determine whether the subject will be responsive to vitamin D therapy based on results obtained from the sample, wherein a polymorphism at one or more loci selected from the group consisting of rs10466589, rs10488699, rs10750097, rs11216103, rs11823543, rs11825181, rs12272004, rs12279373, rs12280724, rs12285095, rs12286037, rs12287066, rs12292921, rs12294259, rs28927680, rs3135506, rs35120633, and rs964184, is indicative of the subject's responsiveness to the vitamin D therapy, and wherein the vitamin D can decrease the triglyceride levels in the subjects having the polymorphism compared to a reference level; and administering the vitamin D to the subjects having the polymorphism.

In one embodiment, the polymorphism is an APOA1-APOA5 polymorphism. In one embodiment, the polymorphism is G/C or C/C polymorphism at the rs3135506 locus. In one embodiment, the polymorphism is in linkage disequilibrium with one or more additional polymorphisms at the one or more loci. In one embodiment, the vitamin D is administrated from 10-10,000 IU. In one embodiment, the reference level is the triglyceride level in individuals without the polymorphism at the one or more loci. In one embodiment, the decrease is at least a 10% decrease. In one embodiment, the sample is a body fluid sample. In one embodiment, the subject is a human patient having or suspected of having hypertriglyceridemia. In one embodiment, the analyzing comprises genotyping the sample using PCR.

In one embodiment, the polymorphism is an APOA1-APOA5 polymorphism. In one embodiment, the polymorphism is A/G or G/G polymorphism at the rs10750097 locus. In one embodiment, the polymorphism is in linkage disequilibrium with one or more additional polymorphisms at the one or more loci. In one embodiment, the vitamin D is administrated from 10-10,000 IU. In one embodiment, the reference level is the triglyceride level in individuals without the polymorphism at the one or more loci. In one embodiment, the decrease is at least a 5% decrease. In one embodiment, the sample is a body fluid sample. In one embodiment, the subject is a human patient having or suspected of having hypertriglyceridemia. In one embodiment, the analyzing comprises genotyping the sample using PCR.

### BRIEF DESCRIPTION OF THE FIGURES

FIGS. 1A-1D are graphs showing the effect of vitamin D on HDL levels in populations and subpopulations with a rs3135506 or rs12272004 polymorphism, as compared to controls. FIG. 1A is a graph showing data from a collective sample of patients with a rs3135506 polymorphism compared to controls. FIG. 1B is a graph showing a collective sample in a subpopulation of patients, during the winter months, that have a rs3135506 polymorphism, as compared to controls. FIG. 1C is a graph showing data from a collective sample of patients with a rs12272004 polymorphism compared to controls. FIG. 1D is a graph showing a collective sample in a subpopulation of patients, during the winter months, that have a rs12272004 polymorphism, as compared to controls.

FIGS. 2A-2B are graphs showing the effect of vitamin D on triglyceride levels in populations and subpopulations with a rs3135506 polymorphism, as compared to controls. FIG. 2A is a graph showing data from a collective sample of patients with a rs3135506 polymorphism compared to controls. FIG. 2B is a graph showing a collective sample in a subpopulation of patients, during the winter months, that have a rs3135506 polymorphism, as compared to controls.

FIGS. 3A-3B are graphs showing sequencing chromatogram and expression assay results, respectively, for APOA5 promoter variants. FIG. 3A is a sequencing chromatogram from vector constructs experiments which illustrate genotypic changes corresponding to SNP rs10750097 from the putative vitamin D receptor binding site. FIG. 3B shows the relative vector expression changes associated with increasing levels of vitamin D. Asterisks indicate significance, as follows: * p<0.05; ** p<0.01; *** p<0.001.

FIGS. 4A-4B are graphs showing vitamin D quintile data for the Intermountain Healthcare Sample (“IHC” sample) population. FIG. 4A shows HDL levels with respect to rs10750097 genotype variants and serum vitamin D levels at 95% confidence intervals. FIG. 4B shows triglyceride levels with respect to rs10750097 genotype variants and serum vitamin D levels at 95% confidence intervals.

### DETAILED DESCRIPTION

The definitions of certain terms as used in this specification are provided below. Unless defined otherwise, all technical and scientific terms used herein generally have the same meaning as commonly understood by one of ordinary skill in the art to which this invention belongs.

As used in this specification and the appended claims, the singular forms “a”, “an” and “the” include plural referents unless the content clearly dictates otherwise. For example, reference to “a nucleic acid” includes a combination of two or more nucleic acids, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

As used herein, the “administration” of an agent or drug to a subject or patient includes any route of introducing or delivering to a subject a compound to perform its intended function. Administration can be carried out by any suitable route, including orally, intranasally, parenterally (intravenously, intramuscularly, intraperitoneally, or subcutaneously), rectally, or topically. Administration includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

As used herein, the terms “amplification” or “amplify” mean one or more methods known in the art for copying a target nucleic acid, e.g., APOA1-APOA5 DNA, thereby increasing the number of copies of a selected nucleic acid sequence. Amplification may be exponential or linear. A target nucleic acid may be either DNA or RNA. The sequences amplified in this manner form an “amplicon.” While the exemplary methods described hereinafter relate to amplification using the polymerase chain reaction (PCR), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al., Eds., Academic Press, San Diego, Calif. 1990, pp. 13-20; Wharam et al., Nucleic Acids Res., 2001, 29(11):E54-E54; Hafner et al., Biotechniques 2001, 30(4):852-6, 858, 860; Zhong et al., Biotechniques, 2001, 30(4):852-6, 858, 860.

As used herein, the term “biomarker” in the context of the present invention refers to a genetic polymorphism, e.g., rs3135506 and/or rs10750097, which is differentially present in a sample taken from one subject as compared to a comparable sample taken from a control subject or a population of control subjects, or as compared to a reference level or value.

The term “clinical factors” as used herein, refers to any data that a medical practitioner may consider in determining a diagnosis or prognosis of disease. Such factors include, but are not limited to, the patient's medical history, a physical examination of the patient, complete blood count, etc.

The terms “comparable” or “corresponding” in the context of comparing two or more samples, mean that the same type of sample, e.g., serum, is used in the comparison. For example, an APOA1-APOA5 polymorphism in a serum sample can be compared to the APOA1-APOA5 polymorphism in another serum sample. In some embodiments, comparable samples may be obtained from the same individual at different times. In other embodiments, comparable samples may be obtained from different individuals, e.g., a patient and a healthy individual. In general, comparable samples are normalized by a common factor. For example, body fluid samples are typically normalized by volume body fluid and cell-containing samples are normalized by protein content or cell count.

The terms “determining,” “measuring,” “assessing,” “analyzing,” “identifying,” and “assaying” are used interchangeably and include both quantitative and qualitative determinations. These terms refer to any form of measurement, and include determining if a characteristic, trait, or feature is present or not. Assessing may be relative or absolute. “Assessing the presence of” includes determining the amount of something present, as well as determining whether it is present or absent.

As used herein, the term “diagnosing” means detecting a disease or disorder, or detecting a factor relating to the disease or disorder, such as, for example, cardiovascular disease, including hyperlipidemia. Diagnosing may also refer to the determination of the stage or degree of a disease or disorder, or a factor relating thereto. Usually, a diagnosis of a disease or disorder is based on the evaluation of one or more factors and/or symptoms that are indicative of the disease. That is, a diagnosis can be made based on the presence, absence, or amount of a factor which is indicative of presence or absence of the disease or condition. Each factor or symptom that is considered to be indicative for the diagnosis of a particular disease does not need be exclusively related to the particular disease, e.g. there may be differential diagnoses that can be inferred from a diagnostic factor or symptom. Likewise, there may be instances where a factor or symptom that is indicative of a particular disease is present in an individual that does not have the particular disease. The term “diagnosing” also encompass determining the therapeutic effect of a drug therapy, or predicting the pattern of response to a drug therapy. The diagnostic methods may be used independently, or in combination with other diagnosing and/or staging methods known in the medical arts for a particular disease or disorder, e.g., hyperlipidemia.

As used herein, the phrase “difference of the level” refers to differences in the quantity of a particular marker, such as a biomarker and/or nucleic acid or protein, in a sample as compared to a control or reference level. For example, the quantity of particular lipoprotein or nucleic acid may be present at an elevated amount or at a decreased amount in samples of patients with a disease compared to a reference level. In one embodiment, a “difference of a level” may be a difference between the level of biomarker present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In one embodiment, a “difference of a level” may be a statistically significant difference between the level of the biomarker present in a sample as compared to a control. For example, a difference may be statistically significant if the measured level of the biomarker falls outside of about 1.0 standard deviations, about 1.5 standard deviations, about 2.0 standard deviations, or about 2.5 stand deviations of the mean of any control or reference group. In another embodiment, a “difference of a level” may be related to levels of serum HDL in a patient before and/or after vitamin D therapy.

As used herein, the term “effective amount” or “pharmaceutically effective amount” or “therapeutically effective amount” of a composition, is a quantity sufficient to achieve a desired therapeutic and/or prophylactic effect, e.g., an amount which results in the prevention of, or a decrease in, the symptoms associated with a disease that is being treated. The amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds and/or treatments.

As used herein, the term “hyperlipidemia,” “hyperlipidemic,” or “hyperlipidemic patient” refers to a condition, or a patient with a condition, that can be classified based on the HDL, LDL, and/or total cholesterol levels in a sample from the patient. For example, HDL cholesterol can be considered “low” if less than 40 mg/dL are detected in a sample. A patient can be considered to have “high” HDL cholesterol, if a sample indicates that the patient has 60 mg/dL or greater HDL. On the other hand, LDL cholesterol can be considered “optimal” when it is present at less than 100 mg/dL. From 100-189 mg/dL, however, LDL cholesterol may be considered “borderline,” whereas LDL levels at 190 mg/dL or greater can be considered “high” or “very high.” With respect to total cholesterol, levels below 200 mg/dL can be considered “optimal,” and levels from 200-239 mg/dL of total cholesterol may be considered “borderline.” Total cholesterol at or in excess of 240 mg/dL may be considered “high.” It may be preferable to increase HDL cholesterol levels, while also, or in the alternative, decreasing the level of LDL cholesterol. Accordingly, “hyperlipidemia” refers to, based on the foregoing classifications, a patient or condition where total and/or HDL cholesterol is less than desirable, and/or where total and/or LDL cholesterol is at a level greater than desirable. Thus, a “hyperlipidemic patient” also refers to subjects that have not been clinically diagnosed as “hyperlipidemic,” but will benefit from the present methods via risk reduction and/or optimization of cholesterol levels.

As used herein, the term “hypertriglyceridemia,” “hypertriglyceridemic,” or “hypertriglyceridemic patient” refers to a condition, or a patient with a condition, that can be classified based on the triglyceride levels in a sample from the patient. For example, triglyceride levels can be considered “low” or “normal” if less than 150 mg/dL are detected in a sample. A patient can be considered to have “borderline” or “borderline-high” triglyceride levels, if a sample indicates that the patient has 151-199 mg/dL or greater triglyceride levels. Furthermore, triglyceride levels can be considered “high” when the levels are from 200-499 mg/dL. Triglyceride levels at or above 500 mg/dL are considered “very high.” A “hypertriglyceridemic patient” also refers to subjects that have not been clinically diagnosed as “hypertriglyceridemic,” but will benefit from the present methods via risk reduction and/or optimization of triglyceride levels.

As used herein, the term “international unit”, or “IU”, used in the context of an amount of vitamin D, refers to a mass-ratio with cholecalciferol, a form of vitamin D produced in the body after exposure to sunlight. In this regard, one IU of vitamin D is equivalent to 0.025 micrograms (μg) of cholecalciferol.

As used herein, “LDL”, “IDL”, “VLDL”, and “HDL” refer to classifications of lipoproteins. It is understood that the values for particle diameter may be determined by gel electrophoresis methods, as known in the art, or mobility analysis methods.

As used herein, the term “linkage disequilibrium” refers to the nonrandom association between two or more alleles such that certain combinations of alleles are more likely to occur together, i.e., they are “linked,” on a chromosome or in a specific genetic locus within a chromosome, compared to other various allele combinations.

As used herein, the term “lipoprotein” refers to constituents particles obtained from mammalian blood which include apolipoproteins biologically assembled with noncovalent bonds to package for example, without limitation, cholesterol and other lipids. Lipoproteins typically refer to biological particles having various sizes, and include very low density lipoproteins (VLDL), intermediate density lipoproteins (IDL), low density lipoproteins (LDL), lipoprotein (a), high density lipoproteins (HDL) and chylomicrons.

As used herein, “microarray” or “gene expression array” or “array” or “tissue microarray” or “polymorphic profile array” refers to an arrangement of a collection of nucleic acids, e.g., nucleotide sequences in a centralized location. Arrays can be on a solid substrate, such as a glass slide, or on a semi-solid substrate, such as nitrocellulose membrane. The nucleotide sequences can be DNA, RNA, or any combination or permutations thereof. The nucleotide sequences can also be partial sequences or fragments from a gene, primers, whole gene sequences, non-coding sequences, coding sequences, published sequences, known sequences, or novel sequences. Tissue microarrays are well known in the art and can be performed as described. See e.g., Camp, R. L., et al., J Clin Oncol, 26, 5630-5637 (2008).

As used herein, “nucleic acid” refers broadly to segments of a chromosome, segments or portions of DNA, cDNA, and/or RNA. Nucleic acid may be derived or obtained from an originally isolated nucleic acid sample from any source, e.g., isolated from, purified from, amplified from, cloned from, or reverse transcribed from sample DNA or RNA. Nucleic acid bases comprise, for example, guanine (G), adenine (A), cytosine (C), thymine (T), and uracil (U), and can include other bases that do not exist naturally in nature, e.g., xanthine.

As used herein, the term “nucleic acid fragment” refers to a sequence of nucleotide residues which are at least about 5 nucleotides, at least about 7 nucleotides, at least about 9 nucleotides, at least about 11, nucleotides, or at least about 17, nucleotides. The fragment is typically less than about 300 nucleotides, less than about 100 nucleotides, less than about 75 nucleotides less than about 50 nucleotides, or less than about 30 nucleotides. In certain embodiments, the nucleic acid fragments can be used in polymerase chain reaction (PCR), or various hybridization procedures to identify or amplify identical or related DNA or RNA molecules.

As used herein, the term “oligonucleotide” refers to a short polymer composed of deoxyribonucleotides, ribonucleotides or any combination thereof. Oligonucleotides are generally between about 10 and about 100 nucleotides in length. Oligonucleotides are typically 15 to 70 nucleotides long, with 20 to 26 nucleotides being the most common. An oligonucleotide may be used as a primer or as a probe. An oligonucleotide is “specific” for a nucleic acid if the oligonucleotide has at least 50% sequence identity with a portion of the nucleic acid when the oligonucleotide and the nucleic acid are aligned. An oligonucleotide that is specific for a nucleic acid is one that, under the appropriate hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, or at least 95% sequence identity.

As used herein, the term “p-value” or “p” refers to a measure of probability that a difference between groups happened by chance. For example, a difference between two groups having a p-value of 0.01 (or p=0.01) means that there is a 1 in 100 chance the result occurred by chance. Suitable p-values include, but are not limited to, 0.1, 0.05, 0.025, 0.02, 0.01, 0.005, 0.001, and 0.0001.

As used herein, the terms “polypeptide,” “peptide,” and “protein” are used interchangeably to refer to a polymer of amino acid residues. The terms apply to amino acid polymers in which one or more amino acid residues is an artificial chemical analog of a corresponding naturally occurring amino acid, as well as to naturally occurring amino acid polymers. The essential nature of such analogs of naturally occurring amino acids is that, when incorporated into a protein, that protein is specifically reactive to antibodies elicited to the same protein but consisting entirely of naturally occurring amino acids. The terms polypeptide, peptide, and protein are also inclusive of modifications including, but not limited to, glycosylation, lipid attachment, sulfation, carboxylation, hydroxylation, ADP-ribosylation, and addition of other complex polysaccharides.

As used herein, a “primer” for amplification is an oligonucleotide that specifically anneals to a target or marker nucleotide sequence. The 3′ nucleotide of the primer should be identical to the target or marker sequence at a corresponding nucleotide position for optimal primer extension by a polymerase. As used herein, a “forward primer” is a primer that anneals to the anti-sense strand of double stranded DNA (dsDNA). A “reverse primer” anneals to the sense-strand of dsDNA.

The term “prognosis” as used herein refers to a prediction of the probable course and outcome of a clinical condition or disease. A prognosis is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. The terms “favorable prognosis” and “positive prognosis,” or “unfavorable prognosis” and “negative prognosis” as used herein are relative terms for the prediction of the probable course and/or likely outcome of a condition or a disease. A favorable or positive prognosis predicts a better outcome for a condition than an unfavorable or negative prognosis. In a general sense, a “favorable prognosis” is an outcome that is relatively better than many other possible prognoses that could be associated with a particular condition, whereas an unfavorable prognosis predicts an outcome that is relatively worse than many other possible prognoses that could be associated with a particular condition. A typical example of a favorable or positive prognosis includes, for example, a longer than expected life expectancy, and the like.

As used herein, the term “reference level” refers to a level of a substance which may be of interest for comparative purposes. In one embodiment, a reference level may be the expression level of a lipoprotein or nucleic acid expressed as an average of the level of the expression level of a protein or nucleic acid from samples taken from a control population of healthy (disease-free) subjects. In another embodiment, the reference level may be the level in the same subject at a different time, e.g., before or after an assay or treatment, such as the level determined prior to the subject developing the disease or prior to initiating therapy. In general, samples are normalized by a common factor. For example, body fluid samples are normalized by volume body fluid and cell-containing samples are normalized by protein content or cell count.

As used herein, the term “sample” or “test sample” refers to any liquid or solid material containing nucleic acids or proteins. In suitable embodiments, a test sample is obtained from a biological source, e.g., a “biological sample”, such as cells in culture or a tissue sample from an animal, most preferably, a human. In an exemplary embodiment, the sample is a serum sample or a whole blood sample.

As used herein, the term “subject” refers to a mammal, such as a human, but can also be another animal such as a domestic animal, e.g., a dog, cat, or the like, a farm animal, e.g., a cow, a sheep, a pig, a horse, or the like, or a laboratory animal, e.g., a monkey, a rat, a mouse, a rabbit, a guinea pig, or the like. The term “patient” refers to a “subject” who is, or is suspected to have a cardiovascular disease or condition, e.g., hyperlipidemia.

As used herein, the term “simultaneous” therapeutic use refers to the administration of at least two active ingredients by the same route and at the same time or at substantially the same time.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the terms “single nucleotide polymorphism,” “SNP”, or “polymorphism” refer to a variation in a nucleotide sequence that occurs when a single nucleotide, e.g., A, T, C, or G, in a genome or other sequence differs between members of a particular species, or when a single nucleotide differs between paired chromosomes within an individual subject or patient. For example, two DNA oligonucleotide fragments from different subjects may contain a difference in a single nucleotide, such as the sequence TTCCT and TTCCG. In such an instance, there are two differing alleles, i.e., the “T allele” and the “G allele.” Typically, SNPs have only two alleles. Moreover, a subject may also be heterozygous or homozygous for a particular SNP. In this case, if the wild-type or naturally occurring allele is “TTC” at a particular locus, and the subject has a sequence of “TTC” on one chromosome at that locus, and TTG on the other paired chromosome, then the subject is said to be heterozygous (C/G) at that locus. However, if the subject has a sequence of “TTG” on one chromosome at the locus, and TTG on the other paired chromosome, then the subject is said to be homozygous (G/G) at that locus. It will be readily understood by the skilled artisan that, for example, because a (C/G) SNP polymorphism on the forward strand also represents the (G/C) SNP polymorphism on the reverse strand, either SNP representation therefore concomitantly imparts the complementary SNP, which may also be referred to as the “rare allele” as used herein.

As used herein, “target nucleic acid” refers to segments of a chromosome, a complete gene with or without intergenic sequence, segments or portions a gene with or without intergenic sequence, or sequence of nucleic acids to which probes or primers are designed. Target nucleic acids may be derived from genomic DNA, cDNA, or RNA. As used herein, target nucleic acid may be native DNA or a PCR-amplified product. In one embodiment, the target nucleic acid is a fragment of a chromosome to be analyzed for methylation, e.g., a promoter region of a gene. In some embodiments, the target nucleic acid is a segment of genomic DNA containing a single nucleotide polymorphism (SNP).

As used herein, the terms “treating” or “treatment” or “alleviation” refers to both therapeutic treatment and prophylactic or preventative measures, wherein the object is to prevent or slow down (lessen) the targeted pathologic condition or disorder. A subject is successfully “treated” for a disorder if, after receiving a therapeutic agent according to the methods of the present disclosure, the subject shows observable and/or measurable reduction in or absence of one or more signs and symptoms of a particular disease or condition.

As used herein, the term “vitamin D” refers to natural and synthetic forms of vitamin D, including, but not limited to, precursors, derivatives, substituted forms, and/or analogs of vitamin D. For example, vitamin D may refer to 25-hydroxyvitamin D or 1,25-dihydroxy-vitamin D, and various forms thereof. Vitamin D may also refer to vitamin D₃, which includes, but is not limited to, e.g., 1α,25-dihydroxyvitamin D₃, 1α,25-dihydroxycholecalciferol, calcitriol, and/or cholecalciferol. Vitamin D may also refer to vitamin D₂, which includes, but is not limited to, e.g., 1α,25-dihydroxyvitamin D₂, ergosterols, and/or ergocalciferol, and the like. In the context of this application, “vitamin D” also refers to vitamin D4, dihydrotachysterol, 22-dihydroergocalciferol, vitamin D5, and/or sitocalciferol.

### Overview

The present disclosure relates to the identification of subject populations for the treatment and management of cardiovascular disease, and conditions or factors related thereto. In particular, hyperlipidemia and hypertriglyceridemia are well known etiological factors that facilitate cardiovascular disease pathogenesis, and remain a significant clinical problem. Accordingly, the present disclosure relates to the identification of subjects that are amenable to therapeutic intervention. The present disclosure further includes methods for treating subjects by administering vitamin D, which may affect a patient's serum triglyceride and/or HDL levels. Methods are described for the detection of single nucleotide polymorphisms (SNPs), including polymorphic variants at the APOA1-APOA5 gene-cluster in order to assist a physician in determining a suitable treatment regime for patients in need thereof. In this regard, when a patient's genetic profile indicates that they are a candidate for treatment, vitamin D is administered to the patient. In one embodiment, vitamin D can decrease serum triglyceride levels and/or increase serum HDL levels in a patient.

Serum HDL levels are influenced by genetic and environmental factors. One nutrient that may influence HDL and Apo-A1 is vitamin D. However, there is no definitive association between an individual's serum vitamin D concentration and their lipid profile. In fact, discrepancies exist as to whether vitamin D correlates with an increase or decrease in serum HDL. See, e.g., Heikkinen et al., Long-term vitamin D₃ supplementation may have adverse effects on serum lipids during postmenopausal hormone replacement therapy. Eur. J. Endocrinol. Vol. 137: 495-502 (1997). Some of the incongruity may relate to varying study designs or differences in patient diet and sun exposure. See Shahar et al., Changes in dietary intake account for seasonal changes in cardiovascular disease risk factors. Eur. J. Clin. Nutr. Vol. 53: 395-400 (1999). The divergent effects of 25-hydroxyvitamin D and 1,25-dihydroxy-vitamin D may also account for conflicting results. See Karhapaa et al., Diverse associations of 25-hydroxyvitamin D and 1,25-dihydroxy-vitamin D with dyslipidaemias. J Intern Med Vol. 24: 1365-2796 (2010). Another source of discrepancy is the gene-environment relationship that is unique to each study population. In this respect, population-specific allele prevalence is one possible reason for the inconsistencies relating to the lipidemic effects of vitamin D.

It has been discovered that there is a relationship between SNP rs10750097 polymorphisms at the APOA1-APOA5 locus and serum vitamin D levels, which is associated with HDL and triglyceride levels in subjects harboring such polymorphisms. Moreover, a correlation between polymorphisms at the rs3135506 APOA1-APOA5 locus and serum vitamin D levels was also identified, which is associated with HDL and triglyceride levels in subjects with such polymorphisms. These associations are even more pronounced during the winter months, and thus, indicate that serum vitamin D mediates HDL and/or triglyceride levels in subjects possessing rs3135506 and/or rs10750097 polymorphisms. One possible mechanism relates to genetic variations that alter the vitamin D receptor binding site.

Although vitamin D receptor binding sites that are capable of influencing gene expression are typically located in promoter regions, functional vitamin D binding sites can also be located in exonic coding regions. See Jiang et al., G2/M arrest by 1,25-dihydroxyvitamin D3 in ovarian cancer cells mediated through the induction of GADD45 via an exonic enhancer. J Biol Chem 278: 48030-48040 (2003). Vitamin D receptor binding sites, moreover, are known to effect promoter expression over 50 kb away. Accordingly, APOA1-APOA5 polymorphisms can potentially influence the expression of any gene at the APOA1-APOA5 locus. See Pike et al., Perspectives on mechanisms of gene regulation by 1,25-dihydroxyvitamin D₃ and its receptor. J Steroid Biochem Mol Biol 103: 389-395 (2007).

Furthermore, in addition to rs3135506, polymorphisms selected from, e.g., rs10466589, rs10488699, rs11216103, rs11823543, rs10750097, rs11825181, rs12272004, rs12279373, rs12280724, rs12285095, rs12286037, rs12287066, rs12292921, rs12294259, rs28927680, rs35120633, and/or rs964184, are in linkage disequilibrium with rs3135506. See, e.g., Teslovich et al., Biological, clinical and population relevance of 95 loci for blood lipids. Nature 466: 707-713 (2010). Likewise, polymorphisms in linkage disequilibrium with rs10750097, e.g., include rs7118999, rs4938303, rs4938302, rs964184, rs11216107, rs11216136 (Caucasian population); rs664059, rs656417, rs2008915, rs3212282, rs10892023, rs10892020, rs180377 (Asian population); and rs9804646, rs11216136, rs61905086, rs12280753, rs2041967 (African population). See Chart A. Accordingly, the foregoing polymorphisms are concomitant biomarkers for subjects that may be responsive to vitamin D therapy.

CHART A

| | | | | | | African Ancestry | | | |
|---|---|---|---|---|---|---|---|---|---|
| European Ancestry | | | | Asian Ancestry | | (Yoruba) | | | |
| | Location | | | | Location | | Location | | |
| SNP | (chr:pos) | r\hat{ }2 | D′ | SNP | (chr:pos) | SNP | (chr:pos) | r\hat{ }2 | D′ |
| rs7118999 | 11:116645275 | 0.62758 | 1 | rs664059 | 11:116642137 | rs9804646 | 11:116665079 | 0.79886 | 1 |
| rs4938303 | 11:116584987 | 0.49061 | 0.92436 | rs656417 | 11:116643436 | rs11216136 | 11:116647120 | 0.53919 | 0.85455 |
| rs4938302 | 11:116584504 | 0.46814 | 0.92259 | rs2008915 | 11:116603134 | rs61905086 | 11:116614863 | 0.48143 | 0.84096 |
| rs964184 | 11:116648917 | 0.45439 | 0.71762 | rs3212282 | 11:116601945 | rs12280753 | 11:116613660 | 0.48143 | 0.84096 |
| rs11216107 | 11:116579663 | 0.44621 | 0.91992 | rs10892023 | 11:116598248 | rs2041967 | 11:116645149 | 0.46929 | 0.71058 |
| rs11216136 | 11:116647120 | 0.4364 | 0.81292 | rs10892020 | 11:116589652 | rs918144 | 11:116633825 | 0.40772 | 0.99999 |
| rs651821 | 11:116662579 | 0.35714 | 0.99999 | rs180377 | 11:116589127 | rs2849179 | 11:116620796 | 0.40772 | 0.99999 |
| rs2072560 | 11:116661826 | 0.35714 | 0.99999 | rs651821 | 11:116662579 | rs180340 | 11:116616402 | 0.40772 | 0.99999 |
| rs57984552 | 11:116566672 | 0.35654 | 0.77087 | rs2072560 | 11:116661826 | rs180362 | 11:116598118 | 0.39358 | 0.99997 |
| rs9804646 | 11:116665079 | 0.30973 | 0.99999 | rs2266788 | 11:116660686 | rs61905088 | 11:116619867 | 0.33116 | 0.79556 |
| rs35606865 | 11:116668843 | 0.30973 | 0.99999 | rs10750096 | 11:116656788 | rs57641217 | 11:116632956 | 0.32874 | 0.79265 |
| rs11216137 | 11:116669828 | 0.30973 | 0.99999 | rs6589566 | 11:116652423 | rs73588403 | 11:116628271 | 0.32874 | 0.79265 |
| rs12294065 | 11:116671540 | 0.30973 | 0.99999 | rs2160669 | 11:116647607 | rs12280724 | 11:116613423 | 0.32874 | 0.79265 |
| rs662799 | 11:116663707 | 0.29515 | 0.85326 | rs662799 | 11:116663707 | rs10466589 | 11:116611985 | 0.32874 | 0.79265 |
| rs7101592 | 11:116668867 | 0.28178 | 0.74633 | rs2075290 | 11:116653296 | rs12274046 | 11:116605070 | 0.28793 | 0.77833 |
| rs12287066 | 11:116662331 | 0.26315 | 0.99999 | rs964184 | 11:116648917 | rs61905078 | 11:116596309 | 0.28793 | 0.77833 |
| rs12274192 | 11:116652351 | 0.26315 | 0.99999 | rs1263151 | 11:116641028 | rs12294259 | 11:116637146 | 0.28512 | 0.77452 |
| rs12286037 | 11:116652207 | 0.26315 | 0.99999 | rs6589565 | 11:116640237 | rs60972755 | 11:116635613 | 0.28512 | 0.77452 |
| rs17120029 | 11:116650118 | 0.26315 | 0.99999 | rs10790162 | 11:116639104 | rs12287066 | 11:116662331 | 0.27969 | 0.90271 |
| rs61905116 | 11:116649538 | 0.26315 | 0.99999 | rs1558861 | 11:116607437 | rs108533 | 11:116601711 | 0.27159 | 0.99997 |
| rs11823543 | 11:116649135 | 0.26315 | 0.99999 | rs3825041 | 11:116631707 | rs12274192 | 11:116652351 | 0.26444 | 0.7648 |
| rs73588403 | 11:116628271 | 0.26315 | 0.99999 | rs7123583 | 11:116600021 | rs12286037 | 11:116652207 | 0.26444 | 0.7648 |
| rs56143464 | 11:116627056 | 0.26315 | 0.99999 | rs6589564 | 11:116624153 | rs17120029 | 11:116650118 | 0.26444 | 0.7648 |
| rs11825181 | 11:116626258 | 0.26315 | 0.99999 | rs180349 | 11:116611827 | rs11823543 | 11:116649135 | 0.26444 | 0.7648 |
| rs57200947 | 11:116623674 | 0.26315 | 0.99999 | rs9326246 | 11:116611733 | rs964184 | 11:116648917 | 0.26444 | 0.7648 |
| rs61905088 | 11:116619867 | 0.26315 | 0.99999 | rs1558860 | 11:116607368 | rs60258347 | 11:116638599 | 0.24066 | 0.99998 |
| rs28927680 | 11:116619073 | 0.26315 | 0.99999 | rs918143 | 11:116630600 | rs79584991 | 11:116636997 | 0.24066 | 0.99998 |
| rs61905086 | 11:116614863 | 0.26315 | 0.99999 | rs2849179 | 11:116620796 | rs78726934 | 11:116620085 | 0.24066 | 0.99998 |
| rs1263056 | 11:116576415 | 0.26153 | 0.99998 | rs180329 | 11:116622958 | rs80165678 | 11:116619849 | 0.24066 | 0.99998 |
| rs656417 | 11:116643436 | 0.25282 | 0.99998 | rs180340 | 11:116616402 | rs75817395 | 11:116619824 | 0.24066 | 0.99998 |
| rs664059 | 11:116642137 | 0.24443 | 0.99996 | rs180346 | 11:116612659 | rs55648563 | 11:116608026 | 0.24066 | 0.99998 |
| rs12791103 | 11:116673315 | 0.24424 | 0.82697 | rs7930786 | 11:116624727 | rs11820589 | 11:116633862 | 0.23864 | 0.80958 |
| rs6589563 | 11:116590787 | 0.24424 | 0.82697 | rs1145208 | 11:116637985 | rs11825181 | 11:116626258 | 0.23864 | 0.80958 |
| rs1263151 | 11:116641028 | 0.23636 | 0.99999 | rs3741301 | 11:116631391 | rs56225305 | 11:116625362 | 0.23864 | 0.80958 |
| rs7103224 | 11:116663966 | 0.21738 | 0.99998 | rs180351 | 11:116607641 | rs12292921 | 11:116621963 | 0.23864 | 0.80958 |
| CM023881 | 11:116662407 | 0.21738 | 0.99998 | rs7118999 | 11:116645275 | rs76341142 | 11:116654531 | 0.23083 | 0.99996 |
| rs12285095 | 11:116658031 | 0.21738 | 0.99998 | rs603446 | 11:116654435 | rs79086998 | 11:116644144 | 0.23082 | 0.99996 |
| rs11600380 | 11:116670182 | 0.21738 | 0.99998 | rs180350 | 11:116610048 | rs57200947 | 11:116623674 | 0.21987 | 0.80037 |
| rs35120633 | 11:116655600 | 0.21738 | 0.99998 | rs4938300 | 11:116567619 | rs28927680 | 11:116619073 | 0.21987 | 0.80037 |
| rs60972755 | 11:116635613 | 0.21738 | 0.99998 | rs3741300 | 11:116631690 | rs7128182 | 11:116666315 | 0.21879 | 0.70742 |
| rs57641217 | 11:116632956 | 0.21738 | 0.99998 | rs180344 | 11:116613184 | rs57232565 | 11:116611457 | 0.20478 | 0.79651 |
| rs56224630 | 11:116627097 | 0.21738 | 0.99998 | rs117935185 | 11:116566538 | rs59455853 | 11:116607874 | 0.20323 | 0.72615 |
| rs56225305 | 11:116625362 | 0.21738 | 0.99998 | rs1787680 | 11:116664776 | rs12285095 | 11:116658031 | 0.20177 | 0.79065 |
| rs61905108 | 11:116624601 | 0.21738 | 0.99998 | rs108533 | 11:116601711 | rs10488699 | 11:116632500 | 0.20177 | 0.79065 |
| rs12292921 | 11:116621963 | 0.21738 | 0.99998 | rs180361 | 11:116598649 | | | | |
| rs12280724 | 11:116613423 | 0.21738 | 0.99998 | rs180365 | 11:116596174 | | | | |
| rs10466533 | 11:116612128 | 0.21738 | 0.99998 | rs180376 | 11:116590039 | | | | |
| rs10466589 | 11:116611985 | 0.21738 | 0.99998 | | | | | | |
| rs59455853 | 11:116607874 | 0.21738 | 0.99998 | | | | | | |
| rs12274146 | 11:116605076 | 0.21738 | 0.99998 | | | | | | |
| rs12274046 | 11:116605070 | 0.21738 | 0.99998 | | | | | | |
| rs12279180 | 11:116604032 | 0.21738 | 0.99998 | | | | | | |
| rs12272004 | 11:116603724 | 0.21738 | 0.99998 | | | | | | |
| rs61905081 | 11:116600564 | 0.21738 | 0.99998 | | | | | | |
| rs12279373 | 11:116600400 | 0.21738 | 0.99998 | | | | | | |
| rs61372904 | 11:116599900 | 0.21738 | 0.99998 | | | | | | |
| rs61905078 | 11:116596309 | 0.21738 | 0.99998 | | | | | | |
| rs11216117 | 11:116596129 | 0.21738 | 0.99998 | | | | | | |
| rs74849419 | 11:116583685 | 0.21738 | 0.99998 | | | | | | |
| rs886022 | 11:116583513 | 0.20677 | 0.99997 | | | | | | |

In one aspect, the present disclosure provides for a method of predicting or identifying whether a patient will respond to vitamin D. As described herein, genetic analyses confirmed the involvement of vitamin D and each of rs3135506 and rs10750097, for example, in relation to serum HDL and triglyceride levels in patients. As such, determining the APOA1-APOA5 allelic genotype at the rs3135506 and/or rs10750097 loci, in a sample from a patient, provides valuable prognostic information for assessing whether the patient will benefit from vitamin D therapy. In some embodiments, vitamin D is administered to a patient having the G/C or C/C genotype, i.e., the heterozygous rare allele and homozygous rare allele, respectively, at the rs3135506 locus. Likewise, vitamin D is administered to a patient having the A/G or G/G genotype, i.e., the heterozygous rare allele and homozygous rare allele, respectively, at the rs10750097 locus, in some embodiments.

The present methods further relate to increasing HDL in a patient by first analyzing a sample from the patient to determine whether the patient will be responsive to vitamin D therapy based on results obtained from the sample. In this respect, a G/C or C/C genotype at the APOA1-APOA5 rs3135506 loci is indicative of the patient's responsiveness to vitamin D. In one embodiment, vitamin D therapy can increase HDL levels in patients that have the G/C or C/C genotype, compared to a reference level. Similarly, an A/G or G/G genotype at the APOA1-APOA5 rs10750097 loci is indicative of the patient's responsiveness to vitamin D. In one embodiment, therefore, vitamin D therapy can increase HDL levels in patients that have the A/G or G/G genotype, compared to a reference level.

In suitable embodiments, it can be determined that a subject will be responsive to vitamin D therapy when results indicate the presence of one or more APOA1-APOA5 biomarkers in a sample, or biomarkers in linkage disequilibrium therewith, e.g., one or more SNPs at the rs10466589, rs3135506, rs10488699, rs10750097, rs11216103, rs11823543, rs11825181, rs12272004, rs12279373, rs12280724, rs12285095, rs12286037, rs12287066, rs12292921, rs12294259, rs28927680, rs35120633, and/or rs964184 loci. In one embodiment, the presence of an APOA1-APOA5 biomarker, or a SNP in linkage disequilibrium with the biomarker, when compared to a reference level, is prognostic for a subject that will respond to vitamin D treatment. In one embodiment, the response is an increase in HDL compared to a reference level. In another embodiment, the response is a decrease in triglyceride levels compared to a reference level. The reference level may be the HDL level and/or level of serum triglycerides in a subject or patient that does not have an APOA1-APOA5 polymorphism.

In addition to the APOA1-APOA5 biomarkers, supplementary diagnostic markers may be combined with a genetic profile to construct models for predicting the presence, absence, or stage of a cardiovascular disease, including prognostic determinations relating thereto. For example, relevant clinical factors for assessing hyperlipidemia or hypertriglyceridemia, including susceptibility to certain treatment regimes, include, but are not limited to, the subject's medical history, a physical examination, complete blood count, and other biological or biochemical markers.

Nevertheless, the APOA1-APOA5 biomarkers described herein are capable of provided a physician with adequate information to determine whether a subject will respond to vitamin D therapy without further assessment or determination. Such biomarkers can be quantitatively or qualitatively determined by empirically or relatively measuring the presence or absence of the biomarkers, in a sample from a subject. The samples include, but are not limited to, sputum, blood (or a fraction of blood such as plasma, serum, or particular cell fractions), lymph, mucus, tears, saliva, urine, semen, ascites fluid, whole blood, and biopsy samples of body tissue. In one embodiment, the sample is a serum sample from a subject that has, or is suspected of having, hyperlipidemia and/or hypertriglyceridemia. In this regard, by employing the present methods, results from a particular sample may reveal that the subject can be efficaciously treated with, for example, vitamin D.

### Sample Collection and Preparation

The methods and compositions described herein may be used to detect nucleic acids associated with various genes using a biological sample obtained from an individual. The nucleic acid (DNA or RNA) may be isolated from the sample according to any methods well known to those of skill in the art. Biological samples may be obtained by standard procedures and may be used immediately or stored, under conditions appropriate for the type of biological sample, for later use.

Starting material for the detection assays is typically a clinical sample, which is suspected to contain the target nucleic acids, e.g., APOA1-APOA5 polymorphic or wild-type DNA. An example of a clinical sample is a blood serum sample. The nucleic acids may be separated from proteins and/or other constituents in the original sample. Any purification methods known in the art may be used in the context of the present invention. Nucleic acid sequences in the sample can successfully be amplified using in vitro amplification, such as PCR. Typically, any compounds that may inhibit polymerases are removed from the nucleic acids.

Methods of obtaining samples are well known to those of skill in the art and include, but are not limited to, aspirations, tissue sections, swabs, drawing of blood or other fluids, surgical or needle biopsies, and the like. The sample may be obtained from an individual or patient. The sample may contain cells, tissues, bone or fluid obtained from a patient suspected being afflicted with a cardiovascular disease. The sample may be a cell-containing liquid or a tissue. Samples may include, but are not limited to, biopsies, bone biopsies, bone marrow biopsies, blood, blood cells, bone marrow, fine needle biopsy samples, plasma, pleural fluid, saliva, serum, tissue or tissue homogenates, frozen or paraffin sections of tissue, and/or oral swabs. Samples may also be processed, such as sectioning of tissues, fractionation, purification, or cellular organelle separation.

If necessary, the sample may be collected or concentrated by centrifugation and the like. The cells of the sample may be subjected to lysis, such as by treatments with enzymes, heat, surfactants, ultrasonication, or a combination thereof. The lysis treatment is performed in order to obtain a sufficient amount of nucleic acid derived from the cells in the sample to detect using polymerase chain reaction.

### Methods for Nucleic Acid Detection

The nucleic acid to be amplified may be from a biological sample such as a blood or serum sample or oral swabs, and the like. Various methods of extraction are suitable for isolating the DNA or RNA. Suitable methods include phenol and chloroform extraction. See Maniatis et al., Molecular Cloning, A Laboratory Manual, 2d, Cold Spring Harbor Laboratory Press, pp. 16-54 (1989). Numerous commercial kits also yield suitable DNA and RNA including, but not limited to, QIAamp™ mini blood kit, Agencourt Genfind™, Roche Cobas® Roche MagNA Pure® or phenol: chloroform extraction using Eppendorf Phase Lock Gels®, and the NucliSens extraction kit (Biomerieux, Marcy l'Etoile, France).

Nucleic acid extracted from cells or tissues can be amplified using nucleic acid amplification techniques well known in the art. By way of example, but not by way of limitation, these techniques can include polymerase chain reaction (PCR), reverse transcriptase polymerase chain reaction (RT-PCR), nested PCR, ligase chain reaction. See Abravaya, K., et al., Nucleic Acids Research, 23:675-682, (1995), branched DNA signal amplification, Urdea, M. S., et al., AIDS, 7 (suppl 2):S11-S14, (1993), amplifiable RNA reporters, Q-beta replication, transcription-based amplification, boomerang DNA amplification, strand displacement activation, cycling probe technology, isothermal nucleic acid sequence based amplification (NASBA). See Kievits, T. et al., J Virological Methods, 35:273-286, (1991), Invader Technology, or other sequence replication assays or signal amplification assays may also be used. Some of these methods of amplification are described briefly below and are well-known in the art.

Some methods employ reverse transcription of RNA to cDNA. The method of reverse transcription and amplification may be performed by previously published or recommended procedures. Various reverse transcriptases may be used, including, but not limited to, MMLV RT, RNase H mutants of MMLV RT such as Superscript and Superscript II (Life Technologies, GIBCO BRL, Gaithersburg, Md.), AMV RT, and thermostable reverse transcriptase from Thermus thermophilus. For example, one method which may be used to convert RNA to cDNA is the protocol adapted from the Superscript II Preamplification system (Life Technologies, GIBCO BRL, Gaithersburg, Md.; catalog no. 18089-011), as described by Rashtchian, A., PCR Methods Applic., 4:S83-S91, (1994).

In one embodiment, PCR is used to amplify a target sequence of interest, e.g., an APOA1-APOA5 SNP or wild-type sequence. PCR is a technique for making many copies of a specific template DNA sequence. The reaction consists of multiple amplification cycles and is initiated using a pair of primer sequences that hybridize to the 5′ and 3′ ends of the sequence to be copied. The amplification cycle includes an initial denaturation, and typically up to 50 cycles of annealing, strand elongation and strand separation (denaturation). In each cycle of the reaction, the DNA sequence between the primers is copied. Primers can bind to the copied DNA as well as the original template sequence, so the total number of copies increases exponentially with time. PCR can be performed as according to Whelan et al., J of Clin Micro, 33(3):556-561 (1995). Briefly, a PCR reaction mixture includes two specific primers, dNTPs, approximately 0.25 U of Taq polymerase, and 1×PCR Buffer.

The skilled artisan is capable of designing and preparing primers that are appropriate for amplifying a target or marker sequences. The length of the amplification primers depends on several factors including the nucleotide sequence identity and the temperature at which these nucleic acids are hybridized or used during in vitro nucleic acid amplification. The considerations necessary to determine a preferred length for an amplification primer of a particular sequence identity are well-known to a person of ordinary skill in the art. For example, the length of a short nucleic acid or oligonucleotide can relate to its hybridization specificity or selectivity. Exemplary primers for detecting APOA1-APOA5 genetic polymorphisms may be designed based on nucleotide sequence data available in the GenBank database.

In some embodiments, the amplification may include a labeled primer or probe, thereby allowing detection of the amplification products corresponding to that primer or probe. In one embodiment, the amplification may include a multiplicity of labeled primers or probes; such primers may be distinguishably labeled, allowing the simultaneous detection of multiple amplification products. In some embodiments, a primer or probe is labeled with a fluorogenic reporter dye that emits a detectable signal. While an appropriate reporter dye may be a fluorescent dye, any reporter dye that can be attached to a detection reagent such as an oligonucleotide probe or primer is suitable for use in the invention. Such dyes include, but are not limited to, Acridine, AMCA, BODIPY, Cascade Blue, Cy2, Cy3, Cy5, Cy7, Edans, Eosin, Erythrosin, Fluorescein, 6-Fam, Tet, Joe, Hex, Oregon Green, Rhodamine, Rhodol Green, Tamra, Rox, and Texas Red.

In another embodiment, the detection reagent may be further labeled with a quencher dye such as Tamra, Dabcyl, or Black Hole Quencher® (“BHQ”), especially when the reagent is used as a self-quenching probe such as a TaqMan®, See U.S. Pat. Nos. 5,210,015 and 5,538,848, or Molecular Beacon probe, See U.S. Pat. Nos. 5,118,801 and 5,312,728, or other stemless or linear beacon probes. See Livak et al., PCR Method Appl., 4:357-362 (1995); Tyagi et al, Nature Biotechnology, 14:303-308 (1996); Nazarenko et al., Nucl. Acids Res., 25:2516-2521 (1997); and U.S. Pat. Nos. 5,866,336 and 6,117,635.

Nucleic acids may be amplified prior to detection or may be detected directly during an amplification step, e.g., “real-time” methods. In some embodiments, the target sequence is amplified using a labeled primer such that the resulting amplicon is detectably labeled. In some embodiments, the primer is fluorescently labeled. In some embodiments, the target sequence is amplified and the resulting amplicon is detected by electrophoresis.

The level of gene expression can be determined by assessing the amount an APOA1-APOA5 polymorphic mRNA in a test sample. Methods of measuring mRNA in samples are known in the art. To measure mRNA levels, the cells in the samples can be lysed and the levels of mRNA in the lysates or in RNA purified or semi-purified from lysates can be measured by any variety of methods familiar to those in the art. Such methods include, without limitation, hybridization assays using detectably labeled DNA or RNA probes, e.g., northern blotting, or quantitative or semi-quantitative RT-PCR methodologies using appropriate oligonucleotide primers. Alternatively, quantitative or semi-quantitative in situ hybridization assays can be carried out using, for example, tissue sections, or unlysed cell suspensions, and detectably labeled, e.g., fluorescent, or enzyme-labeled, DNA or RNA probes. Additional methods for quantifying mRNA include RNA protection assay (“RPA”), cDNA and oligonucleotide microarrays, representation difference analysis (“RDA”), differential display, EST sequence analysis, serial analysis of gene expression (“SAGE”), and multiplex ligation-mediated amplification with the Luminex FlexMAP (“LMF”). See Peck et al., Genome Biol., 7(7):R61 (2006).

Amplification can also be monitored using “real-time” methods. Real time PCR allows for the detection and quantitation of a nucleic acid target. Typically, this approach to quantitative PCR utilizes a fluorescent dye, which may be a double-strand specific dye, such as SYBR Green® I. Alternatively, other fluorescent dyes, e.g., FAM or HEX, may be conjugated to an oligonucleotide probe or a primer. Various instruments capable of performing real time PCR are known in the art and include, for example, ABI Prism® 7900 (Applied Biosystems) and LightCycler® systems (Roche). The fluorescent signal generated at each cycle of PCR is proportional to the amount of PCR product. A plot of fluorescence versus cycle number is used to describe the kinetics of amplification and a fluorescence threshold level is used to define a fractional cycle number related to initial template concentration. When amplification is performed and detected on an instrument capable of reading fluorescence during thermal cycling, the intended PCR product from non-specific PCR products can be differentiated using melting analysis. By measuring the change in fluorescence while gradually increasing the temperature of the reaction subsequent to amplification and signal generation it may be possible to determine the Tₘ of the intended product(s) as well as that of the nonspecific product.

The methods may include amplifying multiple nucleic acids in sample, also known as “multiplex detection” or “multiplexing.” As used herein, the term “multiplex PCR” refers to PCR, which involves adding more than one set of PCR primers to the reaction in order to detect and quantify multiple nucleic acids, including nucleic acids from one or more target gene markers. Furthermore, multiplexing with an internal control, e.g., 18s rRNA, GADPH, or β-actin) provides a control for the PCR without reaction.

In one embodiment, the methods include measuring the presence or absence or level of APOA1-APOA5 polymorphic nucleic acid. Microarrays are an array of distinct polynucleotides or oligonucleotides synthesized on a substrate, such as paper, nylon or other type of membrane, filter, chip, glass slide, or any other suitable solid support. In other embodiments, the microarray is composed of a large number of unique, single-stranded nucleic acid sequences, usually either synthetic antisense oligonucleotides or fragments of cDNAs, fixed to a solid support. Polynucleotides used in the microarray may be oligonucleotides or fragments that are specific to a gene or genes of interest, e.g., APOA1-APOA5 polymorphic DNA.

Fluorescently-labeled single strand (or “first strand”) cDNA probes can be synthesized from total RNA or mRNA by first isolating RNA from the sample of cells to be tested and cells of a control or reference sample. Typically, the two cDNA samples are labeled with different fluorescent dyes, e.g. green Cy3 and red Cy5. The two labeled cDNA samples are mixed and hybridized to the microarray, and the slide is scanned. In the resulting image, the green Cy3 and red Cy5 signals are overlaid—yellow spots indicate equal intensity for the dyes. With the use of image analysis software, signal intensities are determined for each dye at each element of the array, and the logarithm of the ratio of Cy5 intensity to Cy3 intensity is calculated (center). Positive log(Cy5/Cy3) ratios indicate relative excess of the transcript in the Cy5-labeled sample, and negative log(Cy5/Cy3) ratios indicate relative excess of the transcript in the Cy3-labeled sample. Values near zero indicate equal abundance in the two samples. In one embodiment, tissue microarray analysis (“TMA”), can be employed for APOA1-APOA5 polymorphism detection when the sample is a tissue sample or the like. See Camp, R. L., et al., J Clin Oncol, 26, 5630-5307 (2008). In suitable embodiments, next generation sequencing technologies are employed for polymorphism detection. See, e.g., Jarvie, T., Next generation sequencing technologies. Drug Discovery Today: Technologies. Vol. 2 (3) 255-260 (2005). The skilled artisan will readily appreciate that either DNA strand, e.g., the leading or lagging strand, can be employed as a template for SNP detection. Typically, whole genome amplification includes, and therefore amplifies and detects, SNPs in either or both DNA strands.

### Identification and Treatment of Candidate Subjects

In one aspect, the present disclosure provides for the generation of test-sample APOA1-APOA5 polymorphic profiles, which are employed to determine a suitable treatment regime for a hyperlipidemic and/or hypertriglyceridemic patient. If an APOA1-APOA5 polymorphism, e.g., at the rs10750097 and/or rs3135506 loci, is present in the patient's sample then it is likely that the sample is from a patient that will be responsive to therapy, such as vitamin D administration. On the other hand, if an APOA1-APOA5 polymorphism is absent from a sample then it is likely that the sample is from a patient that may not be responsive to vitamin D therapy. In this respect, targeted vitamin D supplementation and/or treatment is beneficial in some embodiments for 15-35% of subjects from European descent, and 40-70% of subjects from African or Asian descent, predicted to harbor vitamin D sensitive alleles. See NCBI, dbSNP build 132. Bethesda, Md.: NCBI (2010).

In one embodiment, the polymorphism is located at the rs3135506, rs10466589, rs10488699, rs10750097, rs11216103, rs11823543, rs11825181, rs12272004, rs12279373, rs12280724, rs12285095, rs12286037, rs12287066, rs12292921, rs12294259, rs28927680, rs35120633, and/or rs964184 loci. See Table 1. The nucleotide base substitutions at these particular loci can be, for example, any base substitution that confers a polymorphism which does not include the wild-type base, thereby constituting a polymorphic profile indicative of a patient that will be responsive to treatment. Such substitutions, include, but are not limited to, G to C, G to A, G to T, C to G, C to A, C to T, A to C, A to G, A to T, T to C, T to A, and/or T to G substitutions. Table 1 illustrates common base substitutions which confer vitamin D sensitivity, e.g., SNPs in linkage disequilibrium with rs3135506 and/or rs10750097.

TABLE 1

| | COMMON | VIT. D SENSITIVE |
|---|---|---|
| SNP | ALLELE | ALLELE |
| RS10466589 | G | A |
| RS10488699 | G | A |
| RS10750097 | A | G |
| RS11216103 | G | A |
| RS11823543 | G | A |
| RS11825181 | G | A |
| RS12272004 | C | A |
| RS12279373 | A | G |
| RS12280724 | G | T |
| RS12285095 | T | G |
| RS12286037 | C | T |
| RS12287066 | C | A |
| RS12292921 | T | G |
| RS12294259 | C | T |
| RS28927680 | G | C |
| RS3135506 | G | C |
| RS35120633 | G | A |
| RS964184 | C | G |
| RS3135506 | G | C |

In one embodiment, the substitution is a G to C substitution at the rs3135506 locus. In other embodiments, the substitution is an A to G substitution at the rs10750097 locus. Moreover, the sample may contain one or more polymorphic substitutions for each specific allele at a particular locus, i.e., a subject may be heterozygous or homozygous for a particular polymorphism. In this respect, when results indicate that a sample contains either a heterozygous G/C or homozygous C/C polymorphism, at the rs3135506 locus, and/or heterozygous A/G or homozygous G/G polymorphism, at the rs10750097 locus, it is likely that the subject will be responsive to vitamin D therapy.

In addition, the present methods allow for the assessment of cardiovascular disease, including hyperlipidemia and/or hypertriglyceridemia, by analyzing a sample from a subject. In this respect, it can be determined whether the treatments provided herein will increase HDL in the subject. In one embodiment, if the subject is a patient with hyperlipidemia and lacks, for example, the G/C or C/C polymorphic genotype at the rs3135506 locus, then the patient is not a likely candidate for vitamin D therapy, which can increase HDL levels in the patient. In another embodiment, if the subject is a patient with hyperlipidemia and has, for example, the G/C or C/C polymorphic genotype at the rs3135506 locus, then the patient is a candidate for vitamin D therapy, wherein an increase in the patient's HDL levels are a likely result, e.g., via administration of vitamin D.

In some embodiments, if the subject is a patient with hyperlipidemia and lacks, for example, the A/G or G/G polymorphic genotype at the rs10750097 locus, then the patient is not a likely candidate for vitamin D therapy, which can increase HDL levels in the patient. In another embodiment, if the subject is a patient with hyperlipidemia and has, for example, the A/G or G/G polymorphic genotype at the rs10750097 locus, then the patient is a candidate for vitamin D therapy, wherein an increase in the patient's HDL levels are a likely result, e.g., via administration of vitamin D.

In another embodiment, if the subject is a patient with hypertriglyceridemia and lacks, for example, the G/C or C/C polymorphic genotype at the rs3135506 locus, then the patient is not a likely candidate for vitamin D therapy, which can decrease triglyceride levels in the patient. However, if the subject is a patient with hypertriglyceridemia and has, for example, the G/C or C/C polymorphic genotype at the rs3135506 locus, then the patient is a candidate for vitamin D therapy, wherein a decrease in the patient's triglyceride levels are a likely result, e.g., via administration of vitamin D.

Furthermore, if the subject is a patient with hypertriglyceridemia and lacks, for example, the A/G or G/G polymorphic genotype at the rs10750097 locus, then the patient is not a likely candidate for vitamin D therapy, which can decrease triglyceride levels in the patient. However, if the subject is a patient with hypertriglyceridemia and has, for example, the A/G or G/G polymorphic genotype at the rs10750097 locus, then the patient is a candidate for vitamin D therapy, wherein a decrease in the patient's triglyceride levels are a likely result, e.g., via administration of vitamin D.

The results from a patient's sample, e.g., the genotype, are reported in an analysis report. In one embodiment, an analysis report is reported to a clinician, other health care provider, epidemiologist, and the like. In another embodiment, an analysis report may include genetic and/or biochemical characterizations of the patient's DNA, HDL levels, and/or lipid profile, in the sample, in addition to other sample characteristics known in the art, e.g., triglycerides, total cholesterol, and/or LDL cholesterol. In one embodiment, reference level or control population levels are also included in the analysis report.

In one aspect, methods for treating hyperlipidemia and/or hypertriglyceridemia include administering effective amounts of vitamin D to patients that have been identified as candidates for such therapy. The methods of the present disclosure further provide treatment regimes that may be selected based upon the presence of an APOA1-APOA5 polymorphism in a sample. In one embodiment, a treatment regime is selected based upon the presence of a SNP listed in Table 1, or any other SNP in linkage disequilibrium with rs10750097 and/or rs3135506.

The methods further include individual or combination therapies employing, in addition to vitamin D, hypolipidemic and/or hypotriglyceridemic agents such as, but not limited to, niacin, statins, fibrates, resins, ezetimibe, phytosterols, orlistat, CETP inhibitors, squalene synthase inhibitors, ApoA-1 milano, AGI-1067, and/or mipomersen. A “hypolipidemic agent” refers to several classes of hypolipidemic drugs, and the like. Hypolipidemic agents may differ in their effect on LDL and/or HDL levels. Typically, hypolipidemic agents may increase HDL levels and/or decrease LDL levels. Similarly, a “hypotriglyceridemic agent” refers to several classes of hypotriglyceridemic drugs, and the like. Hypotriglyceridemic agents may differ in their effect on triglyceride levels. In a clinical setting, the choice of any specific agent will depend on numerous factors, including, but not limited to: the patient's genetic profile, cholesterol or lipid profile, cardiovascular risk, liver and/or kidney function, and other “clinical factors” as described herein.

Individual or combination therapies are beneficial when it is determined, prior to treatment, that particular therapies, e.g., vitamin D, will improve a patient's health or overall survival. Such determinations are based on the present biomarkers, e.g., rs10750097 and/or rs3135506, which include polymorphisms in linkage disequilibrium therewith. In one aspect, the present disclosure includes compositions, which include therapeutic agents and pharmaceutically acceptable carriers therefor.

In this regard, the present disclosure provides for one or more therapeutic agents that are administered to a patient in need thereof. The therapeutic agents can be administered to a patient prior to, during, or after other treatments. In one embodiment, the therapeutic agents are administered to a patient subsequent to determining that the patient is a candidate for such treatment. In this respect, the therapeutic agents are administered to a patient, prior to, or in combination with, treatments for cardiovascular disease, including hyperlipidemia and hypertriglyceridemia.

In one aspect, the therapeutic agents, alone or in combination, are administered to a patient in an effective amount, e.g., a therapeutically effective dose of vitamin D. A therapeutic dose may vary depending upon the type of therapeutic agent, route of administration, and dosage form. Dosage unit forms generally contain between from about 1 mg or International Units (IU) to about 1000 mg or IU of an active ingredient. The preferred composition or compositions is a formulation that exhibits a high therapeutic index. The therapeutic index is the dose ratio between toxic and therapeutic effects which can be expressed as the ratio between LD50 and ED50. The LD50 is the dose lethal to 50% of the population and the ED50 is the dose therapeutically effective in 50% of the population. The LD50 and ED50 are determined by standard pharmaceutical procedures in animal cell cultures or experimental animals.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation.

In the compositions for treating hyperlipidemia or hypertriglyceridemia described herein, vitamin D can be administered at a daily dose from about 0.001-100,000; 5-70,000; 10-50,000; 100-10,000; 200-5,000; 300-1,000; or 400-700 IU. In one embodiment, vitamin D is administered to a patient at a daily dose from about 1-10,000 IU. In another embodiment, vitamin D is administered to a patient at a daily dose from about 400-700 IU. In some embodiments, vitamin D is administered to a patient at a daily dose from about 250-600 IU. In suitable embodiments, vitamin D is administered to a patient alone or in combination with other hypolipidemic and/or hypotriglyceridemic agents, separately, sequentially, or simultaneously, for the treatment of cardiovascular disease, including hyperlipidemia and/or hypertriglyceridemia. In some embodiments, vitamin D is administered more than once a day, e.g., 2, 3, 4, or 5 times daily.

In other embodiments, the therapeutically effective amount of the vitamin D, hypolipidemic agent, and/or hypotriglyceridemic agent can range from about 0.001 mg/kg to about 30 mg/kg body weight of the subject. In some embodiments, the therapeutically effective amount of the agent can range from about 0.05 mg/kg to about 30 mg/kg, from about 0.1 mg/kg to about 30 mg/kg, from about 1 mg/kg to about 25 mg/kg, from about 1 mg/kg to about 20 mg/kg, or from about 1 or 2 mg/kg to about 15 mg/kg.

In one embodiment, the therapeutic agents are hypolipidemic or hypotriglyceridemic agents, such as, but not limited to, niacin, statins, fibrates, resins, ezetimibe, phytosterols, orlistat, CETP inhibitors, squalene synthase inhibitors, ApoA-1 milano, AGI-1067, and/or mipomersen, or any combination thereof. In suitable embodiments, combination therapies are employed, which include vitamin D and one or more hypolipidemic agents. Along these lines, administration of hypolipidemic agents, in concert with vitamin D, may result in an increase in HDL. In one embodiment, vitamin D and analogs thereof are administered to a patient that has been identified as a candidate for treatment. In one embodiment, the patient is administered vitamin D therapy in combination with hypolipidemic agents and/or hypotriglyceridemic.

In this regard, the compositions for treating hyperlipidemia or hypertriglyceridemia include administering vitamin D in combination with hypolipidemic agents, and/or hypotriglyceridemic agents, from about 0.001 IU to about 100,000 IU of a daily dose. In some embodiments, the therapeutically effective amount can range from about 5 IU to about 70,000 IU, from about 10 IU to about 50,000 IU, from about 50 IU to about 10,000 IU, from about 100 IU to about 5000 IU, from about 200 IU to about 1000 IU, or from about 250 IU to about 600 IU of a daily dose.

The therapeutic agents described herein may be administered in a variety of dosage forms. In some aspects, the present disclosure provides for compositions which may be prepared by mixing the therapeutic agents with pharmaceutically acceptable carriers, excipients, binders, diluents or the like to prevent skin cancer. Such compositions can be in the form of, for example, granules, powders, tablets, capsules, syrup, suppositories, injections, emulsions, elixirs, suspensions or solutions. The instant compositions can be formulated for various routes of administration, for example, by oral administration, by topical administration, by nasal administration, by rectal administration, subcutaneous injection, intravenous injection, intramuscular injections, or intraperitoneal injection. The following dosage forms are given by way of example and should not be construed as limiting the instant invention.

Treatment may also include administering the pharmaceutical formulations of the present disclosure in combination with other therapies. For example, the therapeutic agents, compounds, treatments, therapies, drugs, and/or pharmaceutical formulations, of the present disclosure may be administered before, during, or after other conventional therapies or suggested therapeutic protocols. Appropriate combinations and administration regimes can be determined by those of skill in the cardiovascular and/or medicinal arts.

### EXAMPLES

The present disclosure is further illustrated by the following examples, which should not be construed as limiting in any way. The following is a description of the materials and methods used throughout the examples.

Study populations. Factors associated with cardiovascular disease were analyzed using samples from an Aging study population from Utah (“Aging” or “Utah” sample), which included approximately 2500 subjects in 98 Utah pedigrees with 2 or more probands such as, e.g., early stroke, coronary heart disease death, or hypertension. Details regarding subject selection have been previously published. See Williams et al., Recruitment of members of high-risk Utah pedigrees. Control Clin Trials 8: 105S-114S (1987); see also Table 2 below. Additional follow-up assessments were performed, where a validated dietary survey was administered, lipid profiles were determined, DNA was obtained, and standard cardiovascular risk factors were measured. See Hunt et al., Predictors of an increased risk of future hypertension in Utah. A screening analysis. Hypertension 17: 969-976 (1991). In some of the following examples results are described for subjects selected from the foregoing population, wherein samples were excluded if they lacked data relating to complete lipid profiles, genetic polymorphism data, or dietary survey data (n=1060 subjects in 96 families for HDL analysis, and n=1057 subjects in 70 families for triglyceride analysis). See Tables 2-5.

A replication cohort, moreover, consisted of 5243 caucasian subjects from the NHLBI Family Heart Study (“FamHS” or “FHS” sample), which were not previously administered cholesterol modifying medications. Data for these subjects included lipid profiles and genetic analyses, as described. See Heard-Costa et al., NRXN3 is a novel locus for waist circumference: a genome-wide association study from the CHARGE Consortium. PLoS Genet 5: e1000539 (2009). These population-based families were gathered as part of four studies: the Framingham Heart Study, the Utah Family Tree Study, and ARIC centers in Minneapolis and Forsyth County, N.C. (n=2890 individuals in 504 families; see Table 2 for sample characteristics).

Both the Aging and FamHS samples were subdivided by season (see Table 2) when measuring both diet and lipids levels. Winter subsamples included data analyzed from November to March and consisted of 362 Aging subjects and 1006 FamHS subjects as shown below in Table 2. Because HDL and triglyceride levels are inversely correlated, vitamin D-triglyceride interactions were also measured from the foregoing samples, as further described below.

The Intermountain Healthcare Sample (“IHC” sample) population was evaluated by measuring vitamin D serum concentrations in concert with lipid analysis. In this regard, total serum vitamin D, DNA, and other covariates were available for 1650 HDL subjects and 1681 triglyceride subjects pursuant to the Intermountain Healthcare ongoing Angiographic Registry and DNA Bank, which contains patient samples obtained prior to angiography. See Muhlestein, et al., “Usefulness of in-hospital prescription of statin agents after angiographic diagnosis of coronary artery disease in improving continued compliance and reduced mortality.” Am J Cardiol. Vol. 87:257-61 (2001). Post-angiography discharge medication records identified 853 and 866 subjects possessing the included HDL and triglyceride level criteria, respectively, as discussed below. See id. For this subject population, individuals possessing serum vitamin D greater than 40 ng/ml were excluded. Subjects prescribed lipid modifying medications, post-angiography, were also excluded. The final IHC sample cohort therefore included 797 subjects for HDL analysis and 815 for triglyceride analysis. Table 2.

TABLE 2

| | | | | | | | Vitamin D | HDL | LDL | Trigly- | | |
|---|---|---|---|---|---|---|---|---|---|---|---|---|
| | | | | age | sex | BMI | Mean | Mean | Mean | cerides | | |
| | | N | | Mean | % | mean | (SD) | (SD) | (SD) | Mean (SD) | rs3135506 | rs10750097 |
| | | Individuals | Families | (SD) | male | (SD) | IU | mg/dL | mg/dL | mg/dL | MAF | MAF |
| Aging | Total | 1060 | 70 | 45.3 | 47.4 | 28.4 | 355 | 46.5 | 119.5 | 132.6 | 0.065 | 0.223 |
| | Sample | | | (14.3) | | (6.1) | (287) | (12.0) | (28.6) | (81.1) | | |
| | Winter | 362 | 41 | 45.8 | 49.2 | 28.1 | 354 | 48.9 | 119.7 | 126.5 | 0.061 | 0.232 |
| | Sample | | | (14.7) | | (5.8) | (288) | (12.8) | (28.7) | (74.4) | | |
| FamHS | Total | 2890 | 505 | 51.5 | 46.4 | 27.7 | 269 | 49.5 | 124.3 | 150.8 | 0.075 | 0.213 |
| | Sample | | | (13.7) | | (5.5) | (173) | (14.7) | (35.9) | (103.7) | | |
| | Winter | 1006 | 316 | 51.4 | 50.1 | 27.7 | 268 | 49.9 | 124.2 | 152.1 | 0.076 | 0.209 |

| | Sample | | (13.6) | | (5.5) | (174) | (14.7) | (35.0) | (105.8) | | |
|---|---|---|---|---|---|---|---|---|---|---|---|
| | | Individuals | | | | ng/mL | | | | | |
| IHC | Total | 1681 | 64.3 | 66.2 | 28.7 | 23.6 | 40.7 | 104.7 | 188.6 | 0.067 | 0.216 |
| | Sample | | (12.1) | | (6.2) | (6.3) | (13.2) | (36.6) | (175.1) | | |
| IHC | No Med. | 866 | 62.9 | 63.6 | 28.9 | 23.9 | 42.8 | 106.7 | 180.1 | 0.064 | 0.220 |
| | | | (12.6) | | (6.5) | (6.1) | (13.8) | (34.4) | (148.8) | | |

Dietary questionnaire and winter subsamples. Vitamin D intake was measured both in the Aging and FHS samples via a staff-administered semi-quantitative food frequency questionnaire with reproducibility and validity. See, e.g., Willett et al., Reproducibility and validity of a semiquantitative food frequency questionnaire. Am J Epidemiol 122: 51-65 (1985). It was demonstrated that the correlation between serum vitamin D and vitamin D intake from this questionnaire is significant (r=0.29), wherein the highest correlation between dietary and serum vitamin D is during the winter and early spring months (November through March; r=0.65) where sun exposure is less likely to confound data interpretation. See Jacques et al., Comparison of micronutrient intake measured by a dietary questionnaire and biochemical indicators of micronutrient status. Am J Clin Nutr 57: 182-189 (1993).

Accordingly, the Aging and FHS studies were subdivided into seasonal and lipid measurement cohorts, both of which occurred at the same clinic visit for the subject. In particular, the winter subsample (November to March), was employed as a specific cohort because this subpopulation corresponds to the peak association between serum and dietary vitamin D. See Shahar et al. The winter subsample of the Aging study contained 389 subjects, whereas the winter subsample of the FHS study included 1006 subjects. See Table 3.

Study design and analysis for the aging and FHS samples. jPAP software (see Hasstedt et al., jPAP: Document-driven software for genetic analysis. Genet Epidemiol 29 (2005)), was employed for maximum likelihood analysis, correction of potential covariates, and to estimate heritability attributable to gene-environment interaction factors (interaction) measured as changes in estimated polygenic variance. Natural log transformed HDL was employed as the dependent variable, and all maximum likelihood models included age, sex, and log transformed BMI as baseline covariates. Log transformed vitamin D intake, SNP rs3135506, SNP rs10750097, and/or the interactions thereof, were sequentially added to the models for significance determinations relating to each factor. Analyses were performed separately for the entire Aging sample and the Aging winter subsample. The results were confirmed using the FHS sample population.

Data obtained from the sample populations were combined to obtain generalized estimates, wherein the effect sizes for the entire sample included 3949 subjects from 620 families (1368 subjects from the winter subsample). Further, the same covariates included in the FHS sample were added to the Aging study as an additional study population (collective sample). Triglyceride levels were also measured in the collective sample where shown below.

The relative proportion of HDL variance attributable to the individual and interaction effects of the rs3135506 and/or rs10750097 polymorphisms and vitamin D were evaluated using a multiple logistic regression analysis, while correcting for age, sex, and BMI, using R-software. Family structure, using jPAP software, was also analyzed to estimate the relative proportion of heritability that is attributable to the interaction of the rs3135506 and/or rs10750097 polymorphisms and vitamin D on patient HDL levels. These models also included such covariates as: age; sex; and natural log transformed BMI. The magnitude of the combined interaction effect was analyzed using coefficients generated from the collective maximum likelihood models to illustrate the average change in HDL and triglyceride levels in response to variations of vitamin D intake.

Study design and evaluation of the IHC sample population. An increase in vitamin D deficiency during the winter may affect correlations between dietary and serum vitamin D levels. As such, serum vitamin D and lipid measurements were concomitantly measured in the IHC samples to control for confounding correlations relating to seasonal dietary and serum vitamin D concentrations, as well as selection bias, e.g., enriching for vitamin D deficient individuals. In this regard, data was divided into vitamin D quintiles and multiple linear regression models were performed for the entire IHC sample population (and non-medicated subsample) to determine whether the genotypic effects were indeed linear, while concomitantly correcting for age, sex, and BMI.

Lipid profiles. Lipid measurements for the Aging study were obtained between 2004 and 2008 using the vertical auto profile (VAP) method (Atherotech, Birmingham, Ala.). Lipid profiles were obtained for the FHS study as described in Heard-Costa et al.; See also Shirts et al., “Evaluation of the gene-age interactions in HDL cholesterol, LDL cholesterol, and triglyceride levels: The impact of the SORT1 polymorphism on LDL cholesterol levels is age dependent.” Atherosclerosis, Vol. (15):15 (2011).

Genotyping. 2302 subjects from the Aging/Utah study were genotyped via real-time PCR using SimpleProbes™ and genotyping reagents from Idaho Technology (Salt Lake City, Utah). Whole-genome amplification (WGA) genotyping data (FHS study) was performed as described in Heard-Costa et al. Using the NCBI HapMap dataset, SNP rs12272004 perfectly correlates with rs3135506 (r2=1.0). Accordingly, rs12272004 was employed as a control marker for the rs3135506 polymorphism. Three SNPs: rs12272004; rs651821; and rs9804646, moreover, accurately predicted SNP rs10750097 from the 1000 genomes database and were therefore employed as markers for SNP rs10750097. See Consortium GP, “A map of human genome variation from population-scale sequencing.” Nature, Vol. 467: 1061-73 (2010). jPAP software with additive genetic models for maximum likelihood analysis was used to evaluate the SNP rs10750097, in the presence and absence of SNP rs3135506 interaction data, from both the winter-only and full-year samples to determine the best data correlation as detailed below.

Bioinformatic identification of SNPs with respect to putative Vitamin D Receptor (VDR) binding sites. Publically available data sources (Fullerton et al., “The effects of scale: variation in the APOA1/C3/A4/A5 gene cluster.” Hum Genet, Vol. 115:36-56 (2004); Altshuler et al., “Integrating common and rare genetic variation in diverse human populations.” Nature, Vol. 467:52-8 (2010); Wheeler et al., “The complete genome of an individual by massively parallel DNA sequencing.” Nature, Vol. 452:872-6 (2008)) were employed to identify SNPs in linkage disequilibrium (LD) with rs3135506, while CONSITE® software was used to identify putative vitamin D receptor (VDR) binding sites within 40 kb 5′ and 3′ to SNP rs3135506 for both consensus and variant sequences. See, e.g., Altshuler et al., “Integrating common and rare genetic variation in diverse human populations.” Nature, Vol. 467, 52-58 (2010). Candidate SNPs included polymorphic sites where a predicted VDR was present at a TF score, as reported by CONSITE® software, of >65% for at least one variant, i.e., the linked polymorphism improved the consensus match score by at least 1, which indicates a base change that is at least 40% more common in documented VDR binding sites.

Luciferase assays. Renilla luciferase reporter vectors were produced by inserting a 1 kb promoter sequence (GRCh37/hg19:chr11:116,663,024-116,664,075—reverse strand), upstream from the human APOA5 gene containing the predicted VDR site (GenScript®), into pGL4.70 (Promega®). Site directed mutagenesis was employed to generate the reverse strand SNP rs10750097 variant (C/T)—which is the “A/G” forward strand genotype—and assay controls (T/T). The reporter constructs were subsequently co-transfected into HEP3B hepatoma or HEK293 kidney cells, with a control vector (pGL4.10), using lipofectamine 2000 (InVitrogen®). Following transfection, the cells were incubated in MEM containing 10% FBS for 16-24 hours and thereafter supplemented with increasing concentrations of vitamin D (1,25-dihydroxyvitamin D3) dissolved in DMSO for 24 hours, as described. See Wehmeier et al., “Inhibition of apolipoprotein AI gene expression by 1,25-dihydroxyvitamin D3.” Biochim Biophys Acta. Vol. 1737:16-26 (2005). After vitamin D incubation, the cells were lysed and the relative light units (RLUs) were measured using a Veritas Luminometer (Turner Biosystems®). Luciferase activity was normalized to controls for transfection efficiency and analyzed relative to reporter gene expression in the absence of vitamin D supplementation.

### Example 1

### The Influence of Vitamin D on HDL Levels in Populations with SNP rs3135506 (Aging Sample)

Using data obtained from the Aging study population as described above, it was determined that neither vitamin D nor the rs3135506 polymorphism, alone, improved the HDL model. However, the maximum likelihood model was significantly enhanced when both the rs3135506 polymorphism and vitamin D were taken into account for the entire sample population (p=0.005). See Table 3. When the winter subsample was analyzed (Aging study) the correlation was even greater (p=0.0004), compared to the entire sample population, demonstrating that subjects harboring a rs3135506 polymorphism can increase their HDL levels by vitamin D supplementation. Similar results were obtained when the individual effects of vitamin D and rs3135506 were assessed for the winter subsample (p=0.04 and 0.009 respectively). See Table 3.

TABLE 3
ENTIRE SAMPLE

| | HDL | | | | | | TRIGLYCERIDES | |
|---|---|---|---|---|---|---|---|---|
| | | | | | Collective | | Collective | |
| | Aging | | FHS | | sample | | sample | |
| Model | (N = 1060) | | (N = 2889) | | (N = 3949) | | (N = 3948) | |
| Components | likelihood | P value | likelihood | P value | likelihood | P value | likelihood | P value |
| Base | −340.66 | | −222.42 | | −519.28 | | 5866.16 | |
| covariates | | | | | | | | |
| (BC) | | | | | | | | |
| BC and | −343.58 | 0.09 | −222.67 | 0.62 | −519.43 | 0.7 | 5866.16 | 1 |
| Vitamin D | | | | | | | | |
| BC, Vit. D, | −346.2 | 0.11 | −236 | 0.0003 | −534.26 | 0.0001 | 5836.32 | 5 × 10⁻⁸ |
| and rs3135506 | | | | | | | | |
| BC, Vit. D, | −354.27 | 0.005 | −238.84 | 0.09 | −542.03 | 0.005 | 5835.39 | 0.33 |
| and Vit. D | | | | | | | | |
| interaction | | | | | | | | |
| with rs3135506 | | | | | | | | |

WINTER SUBSAMPLE

| | HDL | | | | | | TRIGLYCERIDES | |
|---|---|---|---|---|---|---|---|---|
| | | | | | Collective sample | | Collective sample | |
| | Aging Winter Only | | FHS Winter Only | | Winter Only | | Winter Only | |
| Model | (N = 362) | | (N = 1006) | | (N = 1368) | | (N = 1365) | |
| Components | likelihood | P value | likelihood | P value | likelihood | P value | likelihood | P value |
| Base | −129.54 | | −41.71 | | −156.28 | | 2007.93 | |
| covariates | | | | | | | | |
| (BC) | | | | | | | | |
| BC and | −136.42 | 0.009 | −41.99 | 0.6 | −157.31 | 0.31 | 2007.02 | 0.34 |
| Vitamin D | | | | | | | | |
| BC, Vit. D, | −140.84 | 0.04 | −46.72 | 0.03 | −165.44 | 0.004 | 1986.83 | 0.000007 |
| and rs3135506 | | | | | | | | |
| BC, Vit. D, | −153.55 | 0.0004 | −56.34 | 0.002 | −181.39 | 0.00007 | 1975.88 | 0.0009 |
| and Vit. D | | | | | | | | |
| interaction | | | | | | | | |
| with rs3135506 | | | | | | | | |

### Example 2

### The Influence of Vitamin D on HDL Levels in Populations with SNP rs3135506 (FHS Sample)

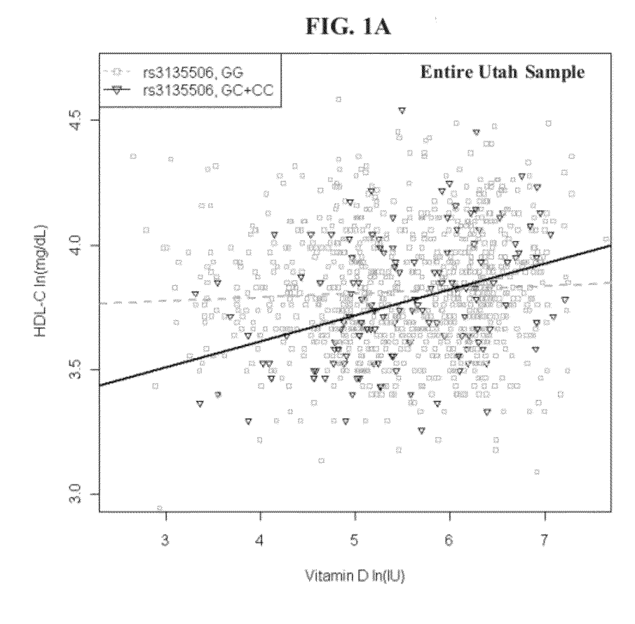

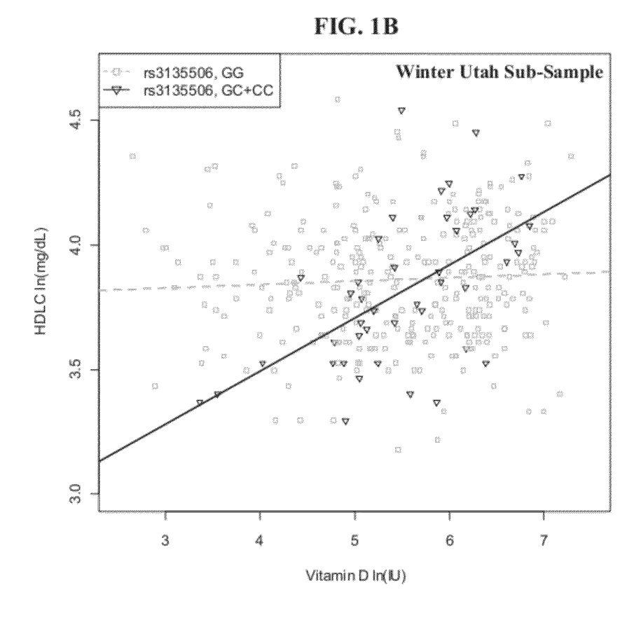

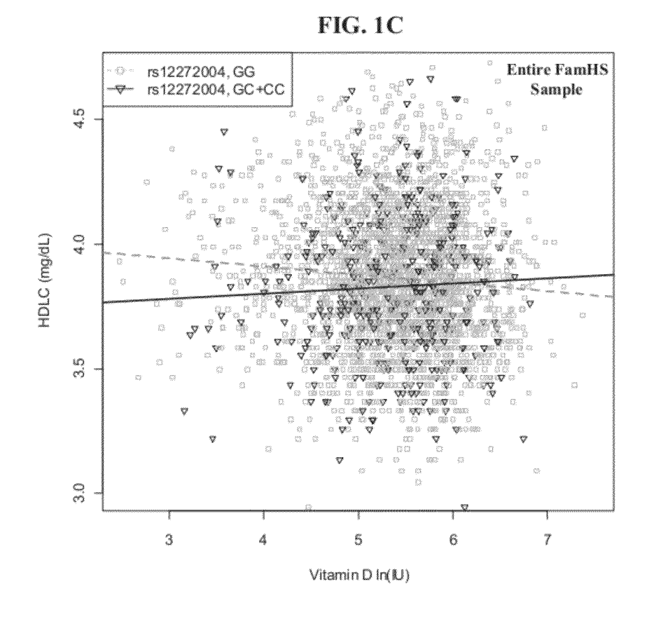

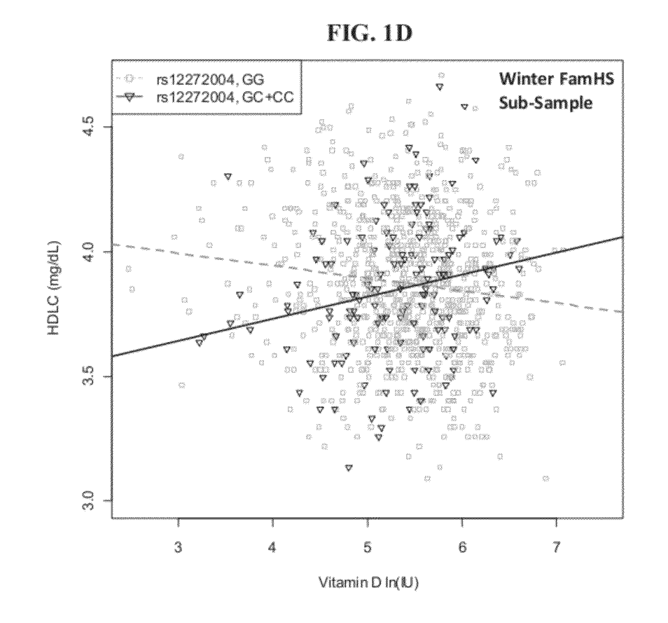

As described above, the FHS study population was also evaluated to confirm data obtained from the Aging study. For this population, when analyzing the entire sample, it was determined that vitamin D, alone, was not associated with an increase in patient HDL levels. However, the rs3135506 polymorphism significantly improved the maximum likelihood model (p=0.0003). See Table 3. Although sample trends were similar for the FHS and Aging population studies (see FIG. 1A-D), the presence of the rs3135506 polymorphism, in combination with vitamin D, did not significantly improve the maximum likelihood model (p=0.09). See Table 3; FIG. 1A-D. Nevertheless, when the FHS winter subsample population was analyzed, it was determined that both the rs3135506 polymorphism, alone, and in combination with vitamin D, significantly contributed to model projections (p=0.03 and p=0.002, respectively). See Table 3.

### Example 3

### Influence of Vitamin D on HDL Levels in Populations with SNP rs3135506 (Collective Sample)

Both FHS and Aging samples populations were also tested as a collective sample. In this regard, the combination of the rs3135506 polymorphism and vitamin D significantly improved the maximum likelihood model for both the entire collective sample (Δ natural log of likelihood=7.77; 1 degree of freedom; p=0.004), and for the collective winter subsample (Δ natural log of likelihood=15.95, 1 degree of freedom; p=0.00007). See Table 3 and FIG. 1A-D.

In accord with these results, for the entire collective sample, the proportion of HDL variance attributable to the rs3135506-vitamin D interaction factor was 0.2%, while the proportion of HDL heritability attributable to the interaction was 0.7%. These results indicate that the interaction of vitamin D with rs3135506 is a more accurate tool for predicting HDL levels in a subject compared to using the genetic locus alone. Furthermore, in the winter collective subsample, the proportion of HDL variance attributable to the rs3135506-vitamin D interaction was 0.9%, while the proportion of HDL heritability attributable to the interaction was 3.3%. As such, based on the estimates from the maximum likelihood models, an increase in wintertime vitamin D intake from 50 IU to 550 IU would decrease HDL cholesterol by approximately 0.5 mg/dL for homozygous G/G subjects. However, subjects that are heterozygous G/C at the rs3135506 locus would increase HDL cholesterol by approximately 10 mg/dL. Moreover, subjects that are homozygous C/C at the rs3135506 locus would increase HDL cholesterol by approximately 20 mg/dL. See FIG. 1A-D.

### Example 4

### Influence of Vitamin D on Triglyceride Levels in Populations with SNP rs3135506 (Collective Sample)

The collective sample study population was also evaluated for a potential association between vitamin D and triglyceride levels in patients harboring a rs3135506 polymorphism. It was determined that rs3135506 is associated with patient triglyceride levels (p=5×10⁻⁸); however, vitamin D did not have a significant effect (and p=0.33). See Tables 3 and 4. Nevertheless, when the collective winter subsample was analyzed, it was determined that both triglyceride levels (p=7×10⁻⁶) and the triglyceride-vitamin D interaction (p=0.0009) were associated with the rs3135506 polymorphism. This association trend, however, has an inverse slope compared to the HDL data trend, i.e., higher levels of vitamin D correlate with lower triglyceride levels in patients that are heterozygous (C/G) for the rs3135506 polymorphism. Homozygous (C/C) patient samples, however, did not possess the same trend in the winter sample. See Tables 3 and 4.

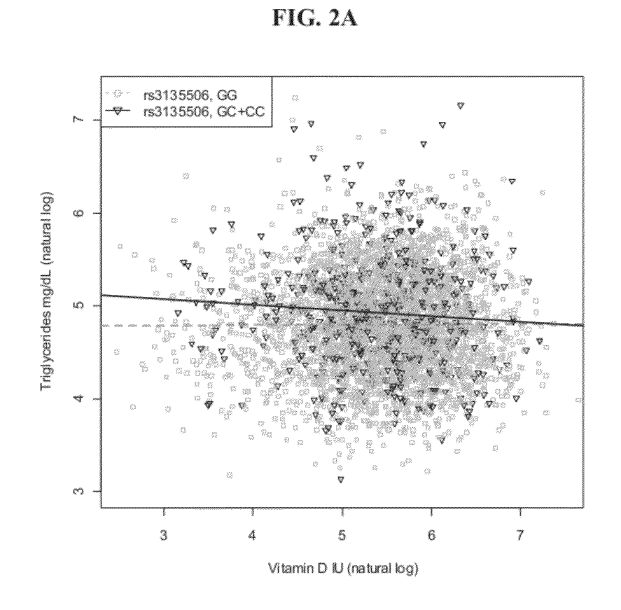

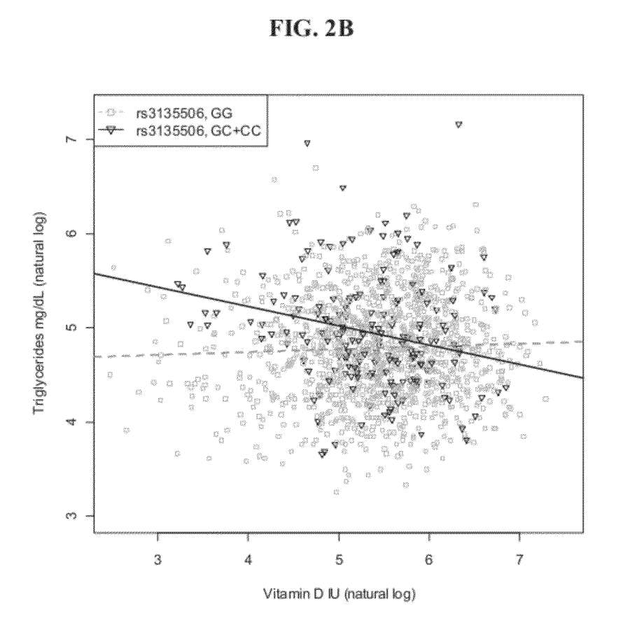

Accordingly, based on the maximum likelihood model, an increase in wintertime vitamin D intake from 50 IU to 550 IU would increase triglycerides by 2 mg/dL in subjects that are homozygous for the G/G rs3135506 allele, i.e., wild-type. However, subjects that are heterozygous G/C at the rs3135506 locus would benefit from vitamin D administration, whereas triglyceride levels would decrease by 50 mg/dL. Moreover, subjects that are homozygous C/C at the rs3135506 locus may also benefit from vitamin D administration, whereas triglyceride levels may decrease by 125 mg/dL. See FIG. 2.

TABLE 4
Maximum Likelihood Models rs3135506 and Triglycerides

| | Aging (N = 1057, 70) | | FHS (N = 2889, 505) | | Combined (N = 3948, 575) | |
|---|---|---|---|---|---|---|
| | | p value for | | p value for | | p value for |
| Model components | Likelihood | difference | Likelihood | difference | Likelihood | difference |
| Base covariates (BC) | 1405.39 | | 4398.99 | | 5866.16 | |
| BC and Vitamin D | 1404.91 | 0.49 | 4398.99 | 0.61 | 5866.16 | 1 |
| BC, Vit. D, and rs3135506 | 1404.85 | 0.81 | 4362.47 | 0.000000002 | 5836.32 | 0.00000005 |
| BC, Vit. D, and Vit. | 1404.77 | 0.78 | 4361.09 | 0.24 | 5835.39 | 0.33 |
| D interaction with | | | | | | |
| rs3135506 | | | | | | |

| | Aging winter only | | FHS winter only | | Combined winter only | |
|---|---|---|---|---|---|---|
| | (N = 359, 41)* | | (N = 1006, 316)* | | (N = 1365,357)* | |
| | | p value for | | p value for | | p value for |
| Model components | Likelihood | difference | Likelihood | difference | Likelihood | difference |
| Base covariates (BC) | 444.39 | | 1546.04 | | 2007.93 | |
| BC and Vitamin D | 441.6 | 0.09 | 1546.01 | 0.86 | 2007.02 | 0.34 |
| BC, Vit. D, and rs3135506 | 437.53 | 0.04 | 1530.2 | 0.00007 | 1986.83 | 0.000007 |
| BC, Vit. D, and Vit. | 436.42 | 0.29 | 1519.26 | 0.0009 | 1975.88 | 0.0009 |
| D interaction with | | | | | | |
| rs3135506 | | | | | | |

N = number of individuals, number of families
BC—baseline covariates are BMI, and sex in aging study with study site added for FHS and combined samples

### Example 5

### Confirmation of VDR Binding Modification Via SNP rs10750097

Using the bioinformatic analyses described above, SNP rs10750097—located 1 kb 5′ to the APOA5 promoter—was predicted to be vitamin D responsive insofar as this variant allele (A/G) increased the VDR binding site score by 1.2, using CONSITE® software with standard parameters. Using CEU samples (subject populations of northern and western European ancestry) from the 1000 Genomes database, the linkage disequilibrium (LD) between SNP rs10750097 and SNP rs3135506 (r2=0.22; D′=1.0) or SNP rs964184 (r2=0.45; D′=0.72), the SNP most commonly reported for this locus, was determined to be significant. It was also determined that rs3741295 affected the VDR binding score by 1.1. This predicted vitamin D responsive allele, however, is associated with the non-vitamin D responsive allele from rs3135506, which therefore indicates that the SNP rs10750097 is likely the causative polymorphism which modifies VDR binding.

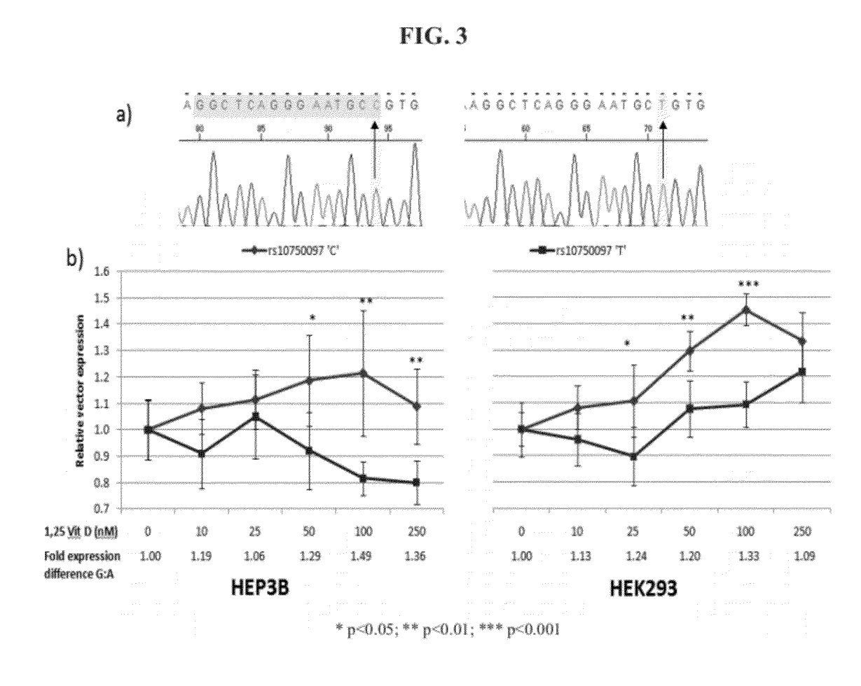

Luciferase assays illustrated that, when compared to the rs10750097 reverse strand “T-allele” (T/T), i.e., the positive strand A/A genotype, APOA5 promoter driven expression was consistently higher for the rs10750097 reverse strand “C-allele”, i.e., the positive strand “G” allele, which would be present at one copy in individuals possessing the A/G genotype and 2 copies in individuals with the G/G genotype, in the presence of 50 nM (1.2 fold) and 100 nM (1.5 fold) of vitamin D (p<0.03 and p<0.002, respectively; see FIG. 3). This difference was significant for both HEP3B and HEK293 data. See FIG. 3A-B.

The SNP rs10750097-VDR interaction was confirmed using the family based sample population studies, as follows. LD between SNP rs3135506 and SNP rs10750097 was similar to data disclosed from public databases, e.g., Ensembl SNP database, (r2=0.25; D′=1.0). In the combined Aging and FamHS samples, SNP rs10750097 was found to be significantly associated with HDL and triglyceride levels (p=0.01 and p=0.001, respectively; see Table 5). Vitamin D-SNP rs10750097 interactions were likewise determined to be significantly associated with HDL and triglyceride levels as shown in Table 5 below (p<0.001 and p=0.04, respectively). The combined winter subsample also showed a positive correlation.

TABLE 5
Maximum Likelihood Models rs10750097

| | Combined HDL | | Combined Triglycerides | |
|---|---|---|---|---|
| | (N = 3138, 596)* | | (N = 3135, 569)* | |
| | | p value for | | p value for |
| Model components | Likelihood | difference | Likelihood | difference |
| Base covariates (BC) | −474.35 | | 4622.33 | |
| BC and Vitamin D | −474.35 | 1.00 | 4622.31 | 0.89 |
| BC, Vit. D, and rs10750097 | −478.27 | 0.01 | 4597.7 | 0.0000007 |
| BC, Vit. D, and Vit. D interaction | −491.81 | 0.00007 | 4593.44 | 0.04 |
| with rs10750097 | | | | |
| | Combined winter only HDL | | Combined winter only Triglycerides | |
| | (N = 1099, 329)* | | (N = 1096, 329)* | |
| | | p value for | | p value for |
| Model components | Likelihood | difference | Likelihood | difference |
| Base covariates (BC) | −158.91 | | 1586.53 | |
| BC and Vitamin D | −158.79 | 0.34 | 1586.41 | 0.29 |
| BC, Vit. D, and rs10750097 | −164.96 | 0.01 | 1567.28 | 0.00001 |
| BC, Vit. D, and Vit. D interaction | −182.81 | 0.00002 | 1561.61 | 0.02 |
| with rs10750097 | | | | |

N = number of individuals, number of families
BC—baseline covariates are BMI, sex, and study site

### Example 6

### Linear Regression Models for SNP rs10750097 Variants

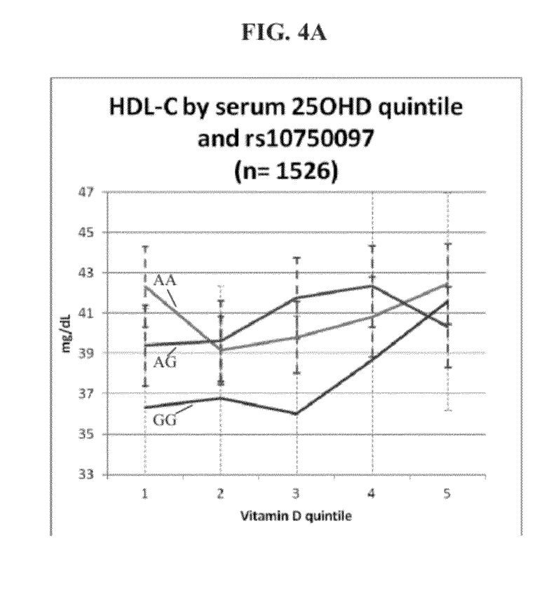

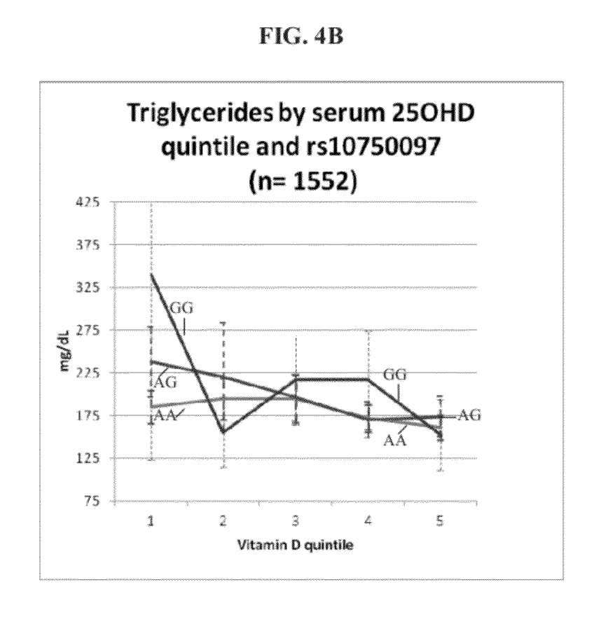

The relationship between SNP rs10750097 genotype variants and HDL or triglyceride levels from the Aging and IHC subject populations were determined as a function of vitamin D concentration. In this respect, while higher levels of dietary vitamin D did not show a significant change, low levels of vitamin D revealed a relationship between different SNP rs10750097 genotypes with respect to HDL levels in the Aging sample. See FIG. 4A-B. This association, however, may not be linear due to the lack of a directly proportional relationship at high vitamin D levels. When IHC samples were examined via linear modeling analyses, no significant correlations were found between SNP rs10750097 genotypes with respect to HDL levels or triglyceride levels (p=0.60 and p=0.76; respectively). Similar results were shown for the non-medicated subsample population (p=0.88 and 0.66, respectively). Data for the IHC subject population nevertheless showed a correlative trend for HDL and triglyceride levels at the lowest vitamin D concentration (see FIG. 4A-B). In accord, lower vitamin D end-point analysis revealed greater genotype-specific differences in HDL levels for rs10750097 variants.

In view of the foregoing results, rs10750097 and rs3135506 were identified as polymorphisms which affect VDR binding, thereby increasing APOA5 promoter activity in vitro. Data from these retrospective studies, moreover, indicates that effective amounts of vitamin D may, for example, increase HDL and decrease triglyceride levels from 5% (heterozygous) and 10% (homozygous) in vitamin D deficient subjects possessing a vitamin D sensitive allele. Consequently, the present inventors have discovered a functional role for vitamin D with respect to inadequate HDL levels and/or high triglyceride levels in subjects possessing the SNP rs10750097 and/or rs3135506 polymorphisms.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present disclosure has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A method for predicting or identifying whether a subject will respond to vitamin D therapy, the method comprising: determining an allelic genotype at one or more loci selected from the group consisting of rs10466589, rs10488699, rs10750097, rs11216103, rs11823543, rs11825181, rs12272004, rs12279373, rs12280724, rs12285095, rs12286037, rs12287066, rs12292921, rs12294259, rs28927680, rs3135506, rs35120633, and rs964184, in a sample from the subject, wherein a polymorphism at the one or more loci is an indication of the subject's responsiveness to the vitamin D therapy.

2. The method of claim 1, wherein the polymorphism is in linkage disequilibrium with one or more additional polymorphisms at the one or more loci.

3. The method of claim 1, wherein the allelic genotype is an APOA1-APOA5 allelic genotype at the rs3135506 loci in the sample from the subject, and the polymorphism is a G/C or C/C polymorphism.

4. The method of claim 3, wherein the G/C or C/C polymorphism indicates that the vitamin D can increase the subject's HDL level compared to a reference level, wherein the reference level is the level of HDL in a sample from an individual without the G/C or C/C polymorphism.

5. The method of claim 3, wherein the G/C or C/C polymorphism indicates that the vitamin D can decrease the subject's triglyceride level compared to a reference level, wherein the reference level is the triglyceride level in a sample from an individual without the G/C or C/C polymorphism.

6. The method of claim 1, wherein the allelic genotype is an APOA1-APOA5 allelic genotype at the rs10750097 loci in the sample from the subject, and the polymorphism is a A/G or G/G polymorphism.

7. The method of claim 6, wherein the A/G or G/G polymorphism indicates that the vitamin D can increase the subject's HDL level compared to a reference level, wherein the reference level is the level of HDL in a sample from an individual without the A/G or G/G polymorphism.

8. The method of claim 6, wherein the A/G or G/G polymorphism indicates that the vitamin D can decrease the subject's triglyceride level compared to a reference level, wherein the reference level is the triglyceride level in a sample from an individual without the A/G or G/G polymorphism.

9. The method of claim 1, further comprising administering the vitamin D to the subjects with the polymorphism at the one or more loci, wherein the vitamin D is administered from 10-10,000 IU.

10. A method for increasing HDL in a subject comprising:
(a) analyzing a sample from the subject to determine whether the subject will be responsive to vitamin D therapy based on results obtained from the sample, wherein a polymorphism at one or more loci selected from the group consisting of rs10466589, rs10488699, rs10750097, rs11216103, rs11823543, rs11825181, rs12272004, rs12279373, rs12280724, rs12285095, rs12286037, rs12287066, rs12292921, rs12294259, rs28927680, rs3135506, rs35120633, and rs964184, is indicative of the subject's responsiveness to the vitamin D therapy, and wherein the vitamin D can increase the HDL level in the subjects having the polymorphism compared to a reference level; and
(b) administering the vitamin D to the subjects having the polymorphism.

11. The method of claim 10, wherein the polymorphism is G/C or C/C polymorphism at the rs3135506 APOA1-APOA5 locus, or A/G or G/G polymorphism at the rs10750097 APOA1-APOA5 locus, or both.

12. The method of claim 10, wherein the polymorphism is in linkage disequilibrium with one or more additional polymorphisms at the one or more loci.

13. The method of claim 10, wherein the vitamin D is administrated from 10-10,000 IU.

14. The method of claim 10, wherein the reference level is the level of HDL in individuals without the polymorphism at the one or more loci.

15. The method of claim 10, wherein the sample is a body fluid sample.

16. A method for decreasing triglyceride levels in a subject comprising:
(a) analyzing a sample from the subject to determine whether the subject will be responsive to vitamin D therapy based on results obtained from the sample, wherein a polymorphism at one or more loci selected from the group consisting of rs10466589, rs10488699, rs10750097, rs11216103, rs11823543, rs11825181, rs12272004, rs12279373, rs12280724, rs12285095, rs12286037, rs12287066, rs12292921, rs12294259, rs28927680, rs3135506, rs35120633, and rs964184, is indicative of the subject's responsiveness to the vitamin D therapy, and wherein the vitamin D can decrease the triglyceride levels in the subjects having the polymorphism compared to a reference level; and
(b) administering the vitamin D to the subjects having the polymorphism.

17. The method of claim 16, wherein the polymorphism is G/C or C/C polymorphism at the rs3135506 APOA1-APOA5 locus, or A/G or G/G polymorphism at the rs10750097 APOA1-APOA5 locus, or both.

18. The method of claim 16, wherein the polymorphism is in linkage disequilibrium with one or more additional polymorphisms at the one or more loci.

19. The method of claim 16, wherein the vitamin D is administrated from 10-10,000 IU.

20. The method of claim 16, wherein the reference level is the triglyceride level in individuals without the polymorphism at the one or more loci.
